(19) United States
(12) Patent Application Publication
(10) Pub. No.: US 2023/0152267 A1
(43) Pub. Date: May 18, 2023

(54) CANCER CELL DETECTION BY MONITORING CHANGES IN PHOTORESPONSE OF GRAPHENE/SILICON SCHOTTKY DIODE

(71) Applicants: Yaser Abdi, Tehran (IR); Anousha Khamsavi, Tehran (IR)

(72) Inventors: Yaser Abdi, Tehran (IR); Anousha Khamsavi, Tehran (IR)

(73) Assignees: Yaser Abdi, Tehran, OT (IR); Anosuha Khamsavi, Tehran, OT (IR); University of Tehran, Tehran (IR)

(21) Appl. No.: 18/099,103

(22) Filed: Jan. 19, 2023

Related U.S. Application Data
(63) Continuation of application No. PCT/IB22/54230, filed on May 6, 2022.
(60) Provisional application No. 63/298,666, filed on Jan. 12, 2022.

(51) Int. Cl. G01N 27/30; H01L 31/108; H01L 31/0216; H01L 31/028; H01L 31/0224

(52) U.S. Cl. CPC G01N 27/30; H01L 31/108; H01L 31/02161; H01L 31/028; H01L 31/022408

(57) ABSTRACT

Disclosed herein is a system for detecting cancer cells. The system includes a biosensor comprising a graphene-Si Schottky junction, a light source placed above the biosensor, an electrical stimulator-analyzer connected to the biosensor, and a processing unit connected to the electrical stimulator-analyzer and the light source. The processing unit is configured to perform a method. The method includes generating a set of photocurrents in a reverse bias regime passed through the graphene-Si Schottky junction with a sample placed thereon utilizing the light source and the electrical stimulator-analyzer, measuring the set of the generated photocurrents through the graphene-semiconductor Schottky junction in reverse bias regime in the presence of the sample utilizing the electrical stimulator-analyzer device, and detecting a presence of cancer cells in the sample responsive to detecting a change in the measured set of the generated photocurrents within the reverse bias regime.

### CROSS-REFERENCE TO RELATED APPLICATION

This application is a continuation-in-part of International Patent Application PCT/IB2022/054230, filed on May 6, 2022, and entitled “CANCER CELL DETECTION BY MONITORING CHANGES IN PHOTORESPONSE OF GRAPHENE/SILICON SCHOTTKY DIODE”, which takes priority from U.S. Provisional Patent Application Ser. No. 63/298,666, filed on Jan. 12, 2022, and entitled “GRAPHENE/SILICON SCHOTTKY JUNCTION BASED DEVICE FOR CANCER CELL ANALYSIS”, which are both incorporated herein by reference in their entirety.

### TECHNICAL FIELD

The present disclosure generally relates to cancer diagnosis, and particularly, to a system and method for real-time diagnosis of cancer cells in a sample via monitoring photocurrent changes of a graphene/silicon schottky junction in the presence of the sample attached to graphene/silicon schottky junction and under light illumination.

### BACKGROUND

Cancer has become one of the greatest challenges of global healthcare. According to the World Health Organization (WHO), cancer is now the second leading cause of death in the world. Glioma, a general term describing primary brain tumors, is the most frequently occurring tumor of central nervous system. Based on the level of malignancy, the World Health Organization (WHO) classifies gliomas as grade I to grade IV. Glioblastoma multiform (GBM) is designated as grade IV and is the most malignant and common primary brain cancer in adults (more than 60%). GBM is known as malignant and invasive cancer with high resistance to various treatments. Most of patients only have 14-15 months of survival after diagnosis and less than 5% will survive for 5 years. In more advanced stages of a diffusely invasive brain cancer, surgery cannot fully remove the invasive cells and this creates recurrence and increased mortality rates. Such an occurrence may be prevented if diagnosis is made possible in the earlier stages of tumor development, where the tumor cannot be visually distinguished. As seen from an increasing number of new cancer cases, and increasing death rates for different cancers of the nervous system, early diagnosis will play a vital role to control, help select the best treatment option, and eventually, decrease the mortality rate of such cancers in the future.

In recent years, nanotechnology has created rapid advances in development of various biosensors based on available solid state devices and nanostructures. Among these, carbon nanostructures (carbon nanotubes, graphene, graphene oxide, etc.) have been widely used as sensing platforms for different biological material. Biocompatibility and excellent electrical properties of these nanostructures, along with their low cost and high sensitivity, has prompted their application as both transducers, where they directly interact with target biomaterial, or templates, where they capture and immobilize biological transducers, such as proteins, DNA, RNA, etc. in various biosensors in different studies. However, most of the proposed sensors for cancer cells detection are suitable for biomarker detection. Whereas, there is a need to analyze cancer cells themselves. Furthermore, reliability and validity of results obtained by biomarker detection are the main problem that limits use of biomarkers as a diagnostic or analyzing variable in a clinical trial or in an epidemiologic study.

Hence, there is a need for a label-free and real-time sensor, method, and system to detect cancer cells. There is also a need for a highly precise and fast approach for detecting cancer cells in a sample acquired from a person suspected to have cancer utilizing a simply fabricated and non-expensive sensor.

### SUMMARY

This summary is intended to provide an overview of the subject matter of this patent, and is not intended to identify essential elements or key elements of the subject matter, nor is it intended to be used to determine the scope of the claimed embodiments. The proper scope of this patent may be ascertained from the claims set forth below in view of the detailed description below and the drawings.

In one general aspect, the present disclosure is directed to a system for detecting Glioblastoma cancer cells. The system may include a biosensor, a light source placed above the biosensor, an electrical stimulator-analyzer device electrically connected to the biosensor, and a processing unit electrically connected to the electrical stimulator-analyzer device and the light source.

In an exemplary embodiment, the biosensor may include a semiconductor layer, an electrically passivating layer, two electrodes, and a graphene layer. In an exemplary embodiment, the semiconductor layer may include a silicon (Si) wafer and electrically passivating layer may include a silicon dioxide (SiO₂) layer coated on a first portion of the Si wafer. In an exemplary embodiment, the two electrodes may include a first electrode deposited on a second portion of the Si wafer and a second electrode deposited on the SiO₂ layer. In an exemplary embodiment, the graphene layer may be coated on parts of the substrate and the second electrode forming a graphene-Si Schottky junction between the Si wafer and the graphene layer. In an exemplary embodiment, a first side of the graphene layer may be in contact with the Si wafer and a second side of the graphene layer may be in contact with the second electrode. In an exemplary embodiment, the graphene-Si Schottky junction may be configured to put a sample thereon. In an exemplary embodiment, the sample may be in contact with the graphene side of the graphene-Si Schottky junction.

In an exemplary embodiment, the light source may include a light emitting device with a wavelength range of 300 nm to 1000 nm. In an exemplary embodiment, the light source may be configured to irradiate a light beam to the graphene-Si Schottky junction with the sample thereon. In an exemplary embodiment, the electrical stimulator-analyzer device may be electrically connected to the two electrodes of the biosensor. In an exemplary embodiment, the stimulator-analyzer device may include an electrical voltage generator and an electrical current sensor. In an exemplary embodiment, the electrical voltage generator may be configured to apply a voltage between the two electrodes and the electrical current sensor may be configured to measure a produced electrical current between the two electrodes responsive to the applied voltage.

In an exemplary embodiment, the processing unit may include a memory having processor-readable instructions stored therein and a processor. In an exemplary embodiment, the processor may be configured to access the memory and execute the processor-readable instructions to configure the processor to perform a method. In an exemplary embodiment, the method may include irradiating a light beam in a range of 500 nm to 900 nm to the graphene-Si Schottky junction with the sample thereon utilizing the light source, applying a first voltage of −1 V and a second voltage of −0.05 V between the two electrodes utilizing the electrical stimulator-analyzer device, measuring a first electrical current generated between the two electrodes responsive to the applied first voltage and a second electrical current generated between the two electrodes responsive to the applied second voltage utilizing the electrical stimulator-analyzer device, and detecting a presence of Glioblastoma cancer cells in the sample if a difference between the first electrical current and the second electrical current is detected to be more than 10 nA.

In an exemplary embodiment, irradiating the light beam to the graphene-Si Schottky junction with the sample thereon may include irradiating the light beam with a wavelength of 850 nm to the graphene-Si Schottky junction with the sample thereon.

In an exemplary embodiment, detecting the presence of Glioblastoma cancer cells in the sample may further include differentiating between T98G Glioblastoma cells and U87 Glioblastoma cells in the sample. In an exemplary embodiment, differentiating between T98G Glioblastoma cells and U87 Glioblastoma cells in the sample may include detecting a presence of T98G Glioblastoma cells in the sample if the first electrical current is less than 1 μA. In another exemplary embodiment, differentiating between T98G Glioblastoma cells and U87 Glioblastoma cells in the sample may include detecting a presence of T98G Glioblastoma cells in the sample if the first electrical current is more than 1 μA.

In an exemplary embodiment, the sample may include a biological sample containing biological cells acquired from a person suspected to have Glioblastoma cancer. In an exemplary embodiment, the sample may include a biopsied sample from a cancer-suspicious mass in body of the person, and combinations thereof.

In an exemplary embodiment, each of the two electrodes may include a gold (Au) film with a thickness in a range of 50 nm to 200 nm. In an exemplary embodiment, the graphene layer may include a monolayer graphene. In an exemplary embodiment, the system may further include a sample holder. In an exemplary embodiment, the sample holder may be placed around the graphene-Si Schottky junction. In an exemplary embodiment, the sample holder may include one or more sidewalls enclosing an area of the graphene-Si Schottky junction with the sample placed thereon. In an exemplary embodiment, the sample holder may be configured to keep the sample on surface of the graphene-Si Schottky junction, prevent the sample from flowing out of the graphene-Si Schottky junction, and prevent entrance of pollutants or external materials to the graphene-Si Schottky junction.

In one more general aspect, the present disclosure is directed to a system for detecting cancer cells. The system may include a biosensor, a light source placed above the biosensor, an electrical stimulator-analyzer device electrically connected to the biosensor, and a processing unit electrically connected to the electrical stimulator-analyzer device and the light source.

In an exemplary embodiment, the biosensor may include a semiconductor layer, an electrically passivating layer, two electrodes, and a graphene layer. In an exemplary embodiment, the electrically passivating layer may be coated on a first portion of the semiconductor layer. In an exemplary embodiment, the two electrodes may include a first electrode deposited on a second portion of the semiconductor layer and a second electrode deposited on the electrically passivating layer. In an exemplary embodiment, the graphene layer may be coated on parts of the semiconductor layer, the electrically passivating layer, and the second electrode forming a graphene-semiconductor Schottky junction between the semiconductor layer and the graphene layer. In an exemplary embodiment, a first side of the graphene layer may be in contact with the semiconductor layer and a second side of the graphene layer may be in contact with the second electrode. In an exemplary embodiment, the graphene-semiconductor Schottky junction may be configured to put a sample thereon.

In an exemplary embodiment, the light source may include a light emitting device with a wavelength range of 300 nm to 1000 nm. In an exemplary embodiment, the light source may be configured to irradiate a light beam to the graphene-semiconductor Schottky junction with the sample thereon. In an exemplary embodiment, the electrical stimulator-analyzer device may be electrically connected to the two electrodes of the biosensor. In an exemplary embodiment, the stimulator-analyzer device may include an electrical voltage generator and an electrical current sensor. In an exemplary embodiment, the electrical voltage generator may be configured to apply a sweeping range of reverse bias voltages between the two electrodes and the electrical current sensor may be configured to measure a set of produced electrical currents between the two electrodes responsive to the applied sweeping range of reverse bias voltages.

In an exemplary embodiment, the processing unit may include a memory having processor-readable instructions stored therein and a processor. In an exemplary embodiment, the processor may be configured to access the memory and execute the processor-readable instructions to configure the processor to perform a method. In an exemplary embodiment, the method may include generating a set of photocurrents in a reverse bias regime through the graphene-Si Schottky junction with the sample placed thereon, measuring the set of generated photocurrents in the reverse bias regime through the graphene-Si Schottky junction with the sample placed thereon, and detecting a presence of cancer cells in the sample if a change in photocurrent within the reverse bias regime is detected.

In an exemplary embodiment, generating the set of photocurrents in the reverse bias regime through the graphene-Si Schottky junction with the sample placed thereon may include irradiating a light beam in a range of 500 nm to 900 nm to the graphene-Si Schottky junction with the sample thereon utilizing the light source and applying a sweeping range of reverse bias voltages between the two electrodes utilizing the electrical stimulator-analyzer device. In an exemplary embodiment, applying the sweeping range of reverse bias voltages between the two electrodes may include applying a set of voltage in a range of −1 V to −0.01 V between the two electrodes.

In an exemplary embodiment, detecting the presence of cancer cells in the sample may include detecting at least two photocurrent values of the measured set of the generated photocurrents being different with each other by a difference magnitude of more than 10 nA.

In an exemplary embodiment, the semiconductor layer may include a silicon (Si) wafer and the electrically passivating layer may include a silicon dioxide (SiO₂) layer coated on a first portion of the Si wafer.

In an exemplary embodiment, the sample may include a biological sample containing biological cells acquired from a person suspected to have cancer. In an exemplary embodiment, the sample may include a biopsied sample from a cancer-suspicious mass in body of the person, and combinations thereof.

In an exemplary embodiment, each of the two electrodes may include a gold (Au) film with a thickness in a range of 50 nm to 200 nm. In an exemplary embodiment, the graphene layer may include a monolayer graphene film. In an exemplary embodiment, the system may further include a sample holder. In an exemplary embodiment, the sample holder may be placed around the graphene-semiconductor Schottky junction. In an exemplary embodiment, the sample holder may include one or more sidewalls enclosing an area of the graphene-semiconductor Schottky junction with the sample placed thereon. In an exemplary embodiment, the sample holder may be configured to keep the sample on surface of the graphene-semiconductor Schottky junction, prevent the sample from flowing out of the graphene-semiconductor Schottky junction, and prevent entrance of pollutants or external materials to the graphene-Si Schottky junction.

### BRIEF DESCRIPTION OF THE DRAWINGS

The drawing figures depict one or more embodiments in accord with the present teachings, by way of example only, not by way of limitation. In the figures, like reference numerals refer to the same or similar elements.

FIG. 1A shows an exemplary system for detecting cancer cells in a sample, consistent with one or more exemplary embodiments of the present disclosure.

FIG. 1B shows an exemplary biosensor with a sample holder, consistent with one or more exemplary embodiments of the present disclosure.

FIG. 1C shows an exploded view of an exemplary sample holder and an exemplary graphene-semiconductor Schottky junction with an exemplary sample thereon, consistent with one or more exemplary embodiments of the present disclosure.

FIG. 2 shows a flow diagram of an exemplary method for fabricating an exemplary biosensor, consistent with one or more exemplary embodiments of the present disclosure.

FIGS. 3A-3I show a schematic view of steps of an exemplary method for fabricating exemplary biosensor, consistent with one or more exemplary embodiments of the present disclosure.

FIG. 4A shows a flow diagram of an exemplary method for detecting cancer cells in a sample, consistent with one or more exemplary embodiments of the present disclosure.

FIG. 4B shows an exemplary flow diagram of an exemplary method for generating an exemplary set of photocurrents in a reverse bias regime through an exemplary graphene-semiconductor Schottky junction, consistent with one or more exemplary embodiments of the present disclosure.

FIG. 4C shows a flow diagram of an exemplary method for detecting Glioblastoma cancer cells in a sample, consistent with one or more exemplary embodiments of the present disclosure.

FIG. 5A shows a schematic representation of a shadow effect regime of operation of an exemplary biosensor, consistent with one or more exemplary embodiments of the present disclosure.

FIG. 5B shows a schematic representation of a charge transfer effect regime of operation of an exemplary biosensor, consistent with one or more exemplary embodiments of the present disclosure.

FIG. 6 shows an exemplary computer system in which an embodiment of the present disclosure, or portions thereof, may be implemented as computer-readable code, consistent with one or more exemplary embodiments of the present disclosure.

FIG. 7 shows a graph that illustrates current-voltage (I-V) characteristics of exemplary fabricated graphene/Si Schottky junction in dark and under illumination by different wavelengths of light, including 380 nm, 425 nm, 520 nm, 620 nm, 740 nm, and 850 nm with the same intensity of about 200 μW/cm², consistent with one or more exemplary embodiments of the present disclosure.

FIG. 8 shows a set of diagrams illustrating current-voltage characteristics of an exemplary biosensor with and without T98G and U87 cell lines as specified in a top guide, in dark and under light illumination with different wavelengths of light, consistent with one or more exemplary embodiments of the present disclosure.

FIG. 9 shows a graph illustrating reverse photocurrent of exemplary biosensor in the absence and presence of different cancer cell lines for different wavelengths at diode voltage (V_{D}) of −1 V, consistent with one or more exemplary embodiments of the present disclosure.

FIG. 10 shows a graph illustrating current-voltage (I-V) characteristics of an exemplary graphene/Si Schottky junction (diode) under illumination with wavelength of 850 nm in the presence of human fibroblast cells and in a bare mode without any cells thereon, consistent with one or more exemplary embodiments of the present disclosure.

FIG. 11 shows a graph illustrating UV-Visible transmission spectra of PBS solutions containing T98G, U87, and human fibroblast cell lines, consistent with one or more exemplary embodiments of the present disclosure.

FIG. 12 shows a set of diagrams illustrating current versus time (I-t) characteristics with and without T98G, U87, fibroblast cells in dark and under illumination with lights of different wavelengths by applying V_{D} of −1 V as specified in a top guide, consistent with one or more exemplary embodiments of the present disclosure.

FIG. 13 shows a chart illustrating relative change of photocurrent versus wavelength with and without different cell lines at V_{D} of −1 V, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

Herein, an exemplary biosensor for cancer cells detection in a biological sample is described. An exemplary biosensor may comprise a mono-layer graphene/(n-type)Si Schottky junction. Furthermore, an exemplary system and method utilizing an exemplary biosensor are described for detection of cancer cells in an exemplary biological sample. An exemplary method may include putting an exemplary biological sample on an exemplary graphene/Si Schottky junction of an exemplary biosensor, irradiating a light beam onto an exemplary graphene/Si Schottky junction with an exemplary biological sample thereon, and monitoring photocurrent of exemplary graphene/Si Schottky junction while applying a reverse bias voltage regime (diode voltage (V_{D}) of less than zero) in the presence of an exemplary biological sample. In an exemplary embodiment, photocurrent of exemplary graphene/Si Schottky junction may be monitored by recording a set of reverse electrical currents respective to an exemplary applied range of reverse voltages. In an exemplary embodiment, each type of cancer cells may show a unique behavior of changes in reverse electrical currents versus an exemplary applied range of reverse voltages. In an exemplary embodiment, an exemplary method may further include comparing an exemplary set of reverse electrical currents versus an exemplary applied range of reverse voltages (an exemplary I-V set) associated with an exemplary biological sample with a plurality of reference I-V sets associated with a respective plurality of cancer cells and detecting a presence of a first-type cancer cells in an exemplary biological sample if an exemplary I-V set is identical to a first reference I-V set associated with the first-type cancer cells. In an exemplary embodiment, the plurality of reference I-V sets associated with the respective plurality of cancer cells may be generated as a calibration dataset for a plurality of cancer cell types utilizing a plurality of biological samples with known cancer type. An exemplary biosensor, system, and method may not only be capable of distinguishing two different cancer cell types, but may also be utilized easily to differentiate cancer cells from healthy human cells.

As used herein, “a reverse electrical current” and “a reverse voltage” may respectively refer to electrical current and electrical voltage in a reverse bias regime of an exemplary graphene/Si Schottky junction. An exemplary graphene/Si Schottky junction comprises an exemplary diode structure having a reverse bias regime and a forward bias regime in a current-voltage (I-V) diagram including a plurality of electrical currents generated within an exemplary graphene/Si Schottky junction versus a respective plurality of applied electrical voltages to an exemplary graphene/Si Schottky junction. In forward bias, a positive terminal of an electrical voltage generator device is connected to p-type or metallic material of an exemplary diode (herein, graphene side) and a negative terminal is connected to n-type material (herein, Si side) so that electrons are injected into n-type material and holes are transferred from Si to the graphene. Whereas, in reverse bias, a reverse process is applied and electrons must overcome the Schottky barrier to reach the conduction band of the n-type semiconductor.

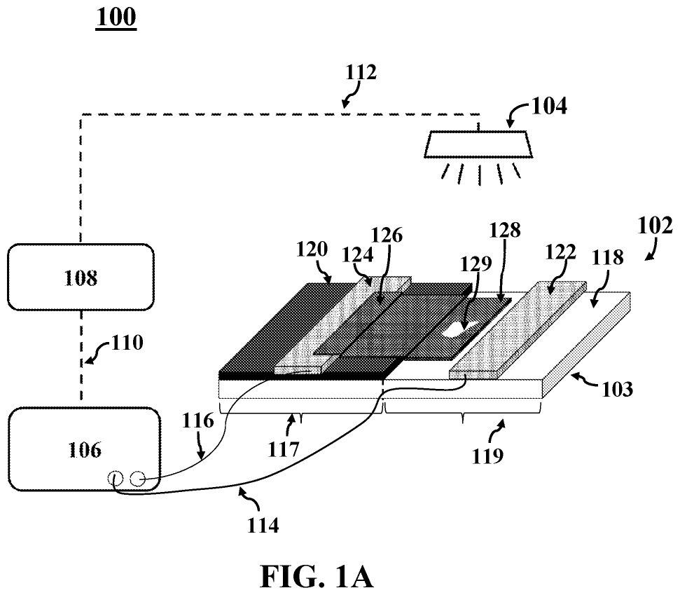

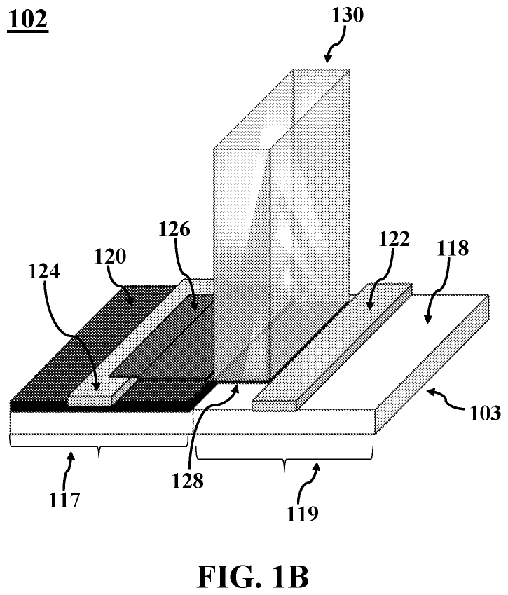

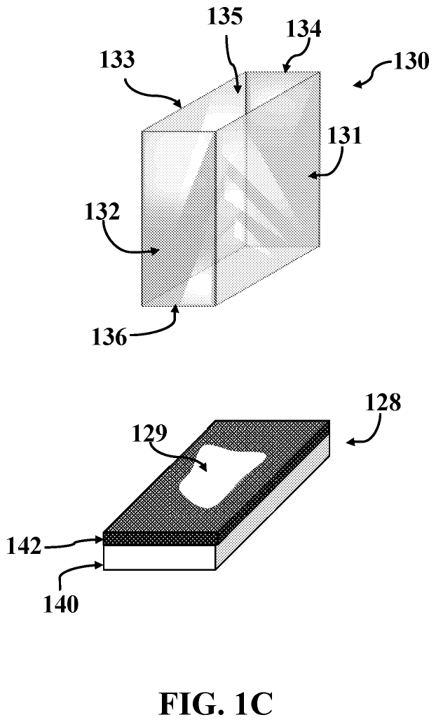

FIG. 1A shows an exemplary system 100 for detecting cancer cells in a sample, consistent with one or more exemplary embodiments of the present disclosure. In an exemplary embodiment, system 100 may include an exemplary biosensor 102, an exemplary light source 104, an exemplary electrical stimulator-analyzer device 106, and an exemplary processing unit 108. In an exemplary embodiment, biosensor 102 may be electrically connected to electrical stimulator-analyzer device 106. In an exemplary embodiment, light source 104 and electrical stimulator-analyzer device 106 may be electrically connected to processing unit 108.

In an exemplary embodiment, an exemplary sample may include a biological sample containing cells. In an exemplary embodiment, an exemplary biological sample may be acquired from a person or an animal suspected to have cancer. In an exemplary embodiment, an exemplary biological sample may include at least one of a blood sample, a biopsied sample from a mass suspicious to be cancerous in a person's body, a cell line, a cell-containing liquid sample drawn from a living body, and combinations thereof. In an exemplary embodiment, an exemplary biological sample may include a sample acquired from a person suspected to have Glioblastoma cancer. As used herein, “Glioblastoma” is an aggressive type of cancer that may occur in brain and/or spinal cord. In an exemplary embodiment, exemplary system 100 may be utilized for detecting Glioblastoma cancer cells in an exemplary sample.

In an exemplary embodiment, biosensor 102 may include a substrate 103, two electrodes 122 and 124, and a graphene layer 126 coated on parts of surface of substrate 103 and two electrodes 122 and 124. In an exemplary embodiment, substrate 103 may include a semiconductor layer 118 and an electrically passivating layer 120 coated on a first portion 117 of semiconductor layer 118. In an exemplary embodiment, first portion 117 of semiconductor layer 118 may include a first half of semiconductor layer 118. In an exemplary embodiment, semiconductor layer 118 may include a silicon (Si) wafer and electrically passivating layer 120 may include a silicon dioxide (SiO₂) layer coated on surface of a first half of an exemplary Si wafer. In an exemplary embodiment, semiconductor layer 118 may include an n-type Si wafer. In an exemplary embodiment, semiconductor layer 118 may have a thickness of about 500 μm. In an exemplary embodiment, electrically passivating layer 120 may have a thickness in a range of about 200 nm to about 1 μm.

In an exemplary embodiment, two electrodes 122 and 124 may include a first electrode 122 deposited on electrically passivating layer 120 and a second electrode 124 deposited on a second portion 119 of semiconductor layer 118. In an exemplary embodiment, each of two electrodes 122 and 124 may include a gold (Au) film with a thickness in a range of about 50 nm to about 200 nm.

In an exemplary embodiment, graphene layer 126 may include a monolayer graphene film coated on parts of surface of electrode 124 and substrate 103. In an exemplary embodiment, graphene layer 126 may cover parts of electrode 124, electrically passivating layer 120, and semiconductor layer 118. In an exemplary embodiment, coated graphene layer 126 may form an exemplary graphene-semiconductor Schottky junction 128 between semiconductor layer 118 and graphene layer 126. In an exemplary embodiment, exemplary graphene-semiconductor Schottky junction 128 may comprise a graphene-Si Schottky junction. In an exemplary embodiment, exemplary graphene-semiconductor Schottky junction 128 may be configured to be able to receive or have put on it an exemplary sample 129. In an exemplary embodiment, a first side of graphene layer 126 may be in contact with semiconductor layer 118 and a second side of graphene layer 126 may be in contact with second electrode 124.

In an exemplary embodiment, biosensor 102 may further include a sample holder. FIG. 1B shows an exemplary biosensor 102 with a sample holder 130, consistent with one or more exemplary embodiments of the present disclosure. In an exemplary embodiment, sample holder 130 may include a hollow enclosure. In an exemplary embodiment, sample holder 130 may include a hollow enclosure with a cross section of one of a circular cross section, a square cross section, a rectangular cross section, a triangular cross section, etc., for example, a hollow enclosure with a rectangular cross section as illustrated in FIG. 1B. In an exemplary embodiment, sample holder 130 may be placed on surface of biosensor 102 around a portion or the entire surface of graphene-semiconductor Schottky junction 128 so that sample holder 130 may enclose a respective portion or the entire surface of graphene-semiconductor Schottky junction 128. In an exemplary embodiment, sample holder 130 may be configured to keep exemplary sample 129 on surface of graphene-semiconductor Schottky junction 128 and prevent exemplary sample 129 from flowing out of graphene-semiconductor Schottky junction 128. For more clarity, FIG. 1C shows an exploded view of sample holder 130 and graphene-semiconductor Schottky junction 128 with exemplary sample 129 thereon, consistent with one or more exemplary embodiments of the present disclosure. In an exemplary embodiment, graphene-semiconductor Schottky junction 128 may comprise a graphene side 142 and a semiconductor side 140. In an exemplary embodiment, graphene side 142 may include a portion of graphene layer 126 coated on semiconductor side 140, where semiconductor side 140 may include a portion of semiconductor layer 118 covered by graphene side 142. In an exemplary embodiment, sample holder 130 may include a hollow cuboid with two open sides 135 and 136 respectively at top and bottom of sample holder 130. In an exemplary embodiment, sample holder 130 may include one or more sidewalls, for example, sidewalls 131, 132, 133, and 134, enclosing a portion or the entire surface of graphene-semiconductor Schottky junction 128 In an exemplary embodiment, exemplary sample holder 334 may be made of plexiglass. In an exemplary embodiment, plexiglass may refer to a plastic material made from poly methyl methacrylate.

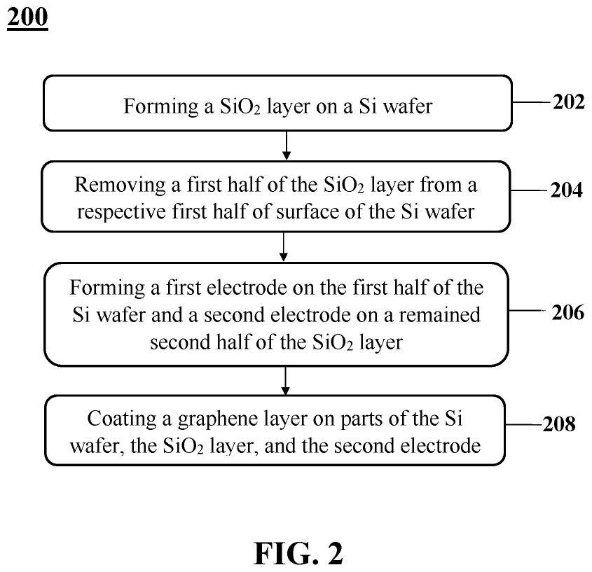

In another general aspect of the present disclosure, a method for fabricating an exemplary biosensor 102 is described. FIG. 2 shows a flow diagram of an exemplary method 200 for fabricating exemplary biosensor 102, consistent with one or more exemplary embodiments of the present disclosure. In an exemplary embodiment, exemplary method 200 of fabricating exemplary biosensor 102 may include forming a SiO₂ layer on a Si wafer (step 202), removing a first half of the SiO₂ layer from a respective first half of surface of the Si wafer (step 204), forming a first electrode on the first half of the Si wafer and a second electrode on a remaining second half of the SiO₂ layer (step 206), and coating a graphene layer on parts of the Si wafer, the SiO₂ layer, and the second electrode (step 208).

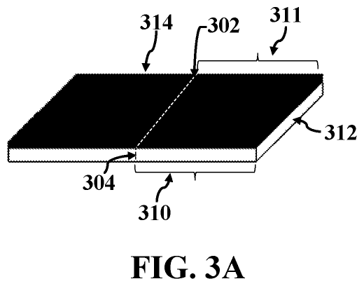

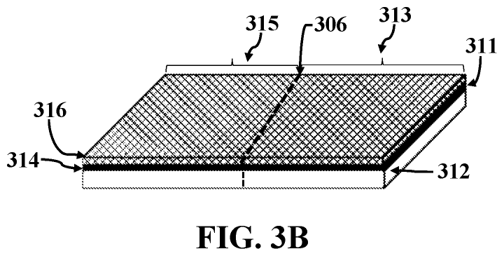

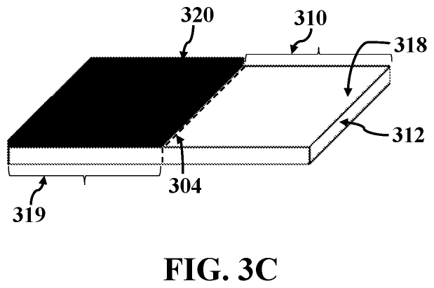

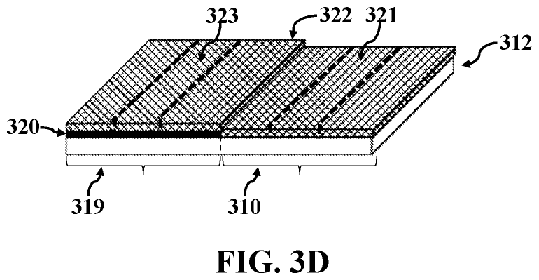

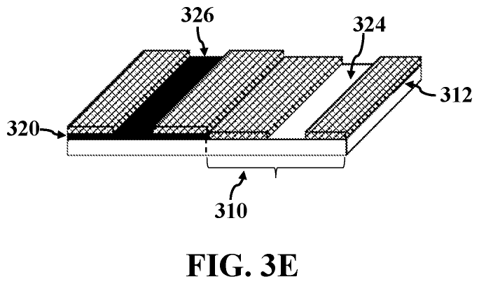

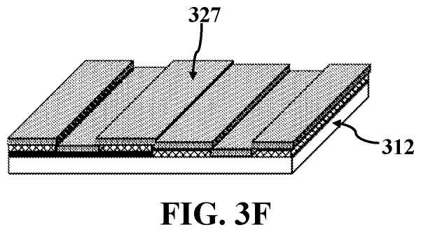

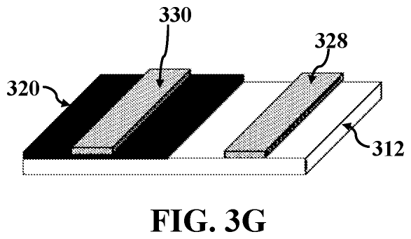

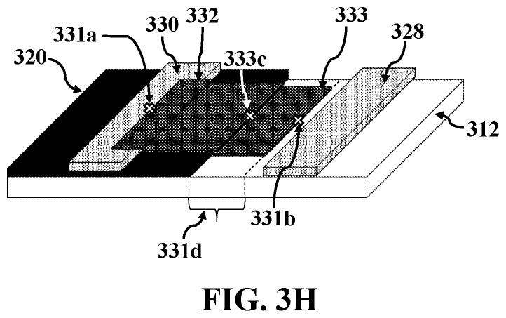

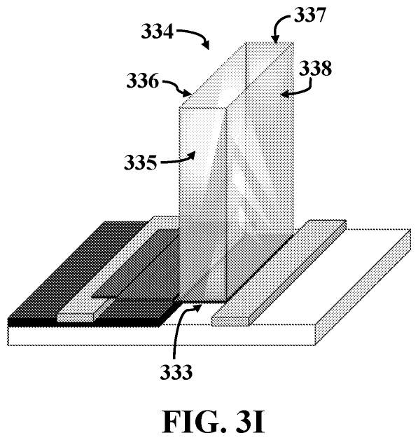

Furthermore, FIGS. 3A-3I shows a schematic view of steps of exemplary method 200 for fabricating exemplary biosensor 102, consistent with one or more exemplary embodiments of the present disclosure. In detail, in step 202, a SiO₂ layer 314 may be formed on a Si wafer 312 as illustrated in FIG. 3A. In an exemplary embodiment, step 202 may include cleaning an exemplary Si wafer 312 as an example of semiconductor layer 118 and forming an exemplary SiO₂ layer 314 as an example of electrically passivating layer 120 on surface of exemplary Si wafer 312. In an exemplary embodiment, Si wafer 312 may include an n-type Si wafer. In an exemplary embodiment, Si wafer 312 may be cleaned via a standard RCA cleaning method. In an exemplary embodiment, electrically insulating SiO₂ layer 314 may be grown on whole surface of Si wafer 312 using thermal oxidation in a quartz furnace. In an exemplary embodiment, Si wafer 312 may be placed in a quartz furnace, temperature may be slowly ramped up to about 1100° C. inside the quartz furnace, and Si wafer 312 may be held therein for oxidation under a constant flow of O₂ gas; thereby, resulting in forming an oxide layer on Si wafer 312 after a time period in a range of 20 minutes to 1 hour.

In an exemplary embodiment, step 204 may include removing a first half 311 of SiO₂ layer 314 from surface of a respective first half 310 of Si wafer 312 considering a hypothetical symmetric line 302 at the middle of SiO₂ layer 314 and a respective hypothetical symmetric line 304 at the middle of Si wafer 312. In an exemplary embodiment, exemplary first half 311 of exemplary SiO₂ layer 314 may be removed using a standard photolithography technique through a two-step process shown in FIGS. 3B and 3C. In an exemplary embodiment, removing exemplary first half 311 of SiO₂ layer 314 may include coating a first layer 316 of a photoresist material on SiO₂ layer 314 as illustrated in FIG. 3B, removing exemplary first half 311 of SiO₂ layer 314 along with a respective first half 313 of first layer 316 of the photoresist material thereon (considering a hypothetical symmetric line 302 at the middle of first layer 316 of the photoresist material) via a photolithography technique, and removing a remaining second half 315 of first layer 316 of photoresist material remained on a respective second half 320 of SiO₂ layer 314 as illustrated in FIG. 3C. In an exemplary embodiment, first layer 316 of the photoresist material may cover the entire top surface of SiO₂ layer 314. In an exemplary embodiment, the photoresist material may include a cured negative photoresist polymer. In an exemplary embodiment, exemplary first half 311 of SiO₂ layer 314 along with a respective first half 313 of first layer 316 of the photoresist material thereon may be removed by etching and/or patterning SiO₂ layer 314 with first layer 316 of photoresist material thereon through a photolithography process. In an exemplary embodiment, remaining second half 315 of the photoresist material after applying photolithography technique may be removed using a NaOH solution. In an exemplary embodiment, exemplary first half 311 of SiO₂ layer 314 may be removed using a 10% Hydrofluoric acid (HF) solution through the photolithography process. In an exemplary embodiment, applying step 204 may lead to forming exemplary first half 310 of exemplary Si wafer 312 having a bare surface 318 and exemplary second half 320 of SiO₂ layer 314 remaining on a respective second half 319 of exemplary Si wafer 312 considering hypothetical symmetric line 304 at the middle of exemplary Si wafer 312 as illustrated in FIG. 3C.

Furthermore, step 206 may include forming two exemplary electrodes similar to electrodes 122 and 124. In an exemplary embodiment, step 206 may include forming an exemplary first electrode on first half 310 of Si wafer 312 with bare surface 318 and forming an exemplary second electrode on remaining second half 320 of SiO₂ layer 314. In an exemplary embodiment, step 206 may be done through four steps schematically illustrated in FIGS. 3D-3G. In an exemplary embodiment, step 206 may include coating a second layer 322 of the photoresist material on bare surface 318 of first half 310 of Si wafer 312 and on remaining second half 320 of SiO₂ layer 314 as illustrated in FIG. 3D. Moreover, step 206 may include forming two electrode sites 324 and 326 by removing two respective middle parts 321 and 323 of second layer 322 of the photoresist material as illustrated in FIGS. 3D and 3E. In an exemplary embodiment, two electrode sites 324 and 326 may be formed by etching and/or patterning second layer 322 of the photoresist material using a standard photolithography technique. In an exemplary embodiment, two electrode sites 324 and 326 may include two openings through second layer 322 of the photoresist material, including a first electrode site 324 on surface of first half 310 of Si wafer 312 and a second electrode site 326 on surface of second half 320 of SiO₂ layer 314. Step 206 may additionally include forming two electrodes 328 and 330 on respective two electrode sites 324 and 326 as illustrated in FIGS. 3F and 3G. In an exemplary embodiment, forming two electrodes 328 and 330 may include coating an exemplary Au film 327 on remaining parts of second layer 322 of the photoresist material and two electrode sites 324 and 326 using a physical vapor deposition (PVD) technique and etching/patterning coated Au film 327 using a standard photolithography technique to form two electrodes 328 and 330 similar to two exemplary electrodes 122 and 124 of exemplary biosensor 102 of FIG. 1. In an exemplary embodiment, coated Au film 327 may have a thickness in a range of about 50 nm to 200 nm. In an exemplary embodiment, step 206 may further include etching all remaining parts of second layer 322 of the photoresist material away using acetone or a NaOH solution so that the photoresist material may be completely removed from structure of an exemplary fabricated biosensor. In an exemplary embodiment, an exemplary fabricated biosensor may be rinsed in acetone to remove the photoresist material before conducting step 208.

Moving to step 208, a graphene-Si Schottky junction similar to graphene-semiconductor Schottky junction 128 may be formed by coating a graphene layer on parts of surface of Si wafer 312, remaining second half 320 of SiO₂ layer 314, and second electrode 330. In an exemplary embodiment, step 208 may include coating an exemplary graphene layer 332 on parts of surface of Si wafer 312, remaining second half 320 of SiO₂ layer 314, and second electrode 330 as illustrated in FIG. 3H. In an exemplary embodiment, exemplary graphene layer 332 may include a layer of graphene coated from a point 331a on surface of second electrode 330 to a point 331b on surface of Si wafer 312; thereby, forming a graphene-Si Schottky junction 333 (a graphene/Si bilayer 333) between graphene layer 332 and Si wafer 312. In an exemplary embodiment, graphene-Si Schottky junction 333 (graphene/Si bilayer 333) may comprise a portion of graphene layer 332 on Si wafer 312 from points 331c to 331b and a respective portion of Si wafer 312 in a zone 331d between points 331c to 331b covered with graphene layer 332. In an exemplary embodiment, graphene layer 332 may include a monolayer graphene film deposited on parts of surface of Si wafer 312, SiO₂ layer 314, and second electrode 330.

In an exemplary embodiment, exemplary method 200 may further include placing an exemplary sample holder 334, similar to exemplary sample holder 130, around a portion or the entire surface of graphene-Si Schottky junction 333 as illustrated in FIG. 31. In an exemplary embodiment, exemplary sample holder 334 may enclose a portion or the entire surface of graphene-Si Schottky junction 333; allowing for keeping an exemplary sample, similar to sample 129, on surface of graphene-Si Schottky junction 333, preventing an exemplary sample from flowing out of graphene-Si Schottky junction 333, and preventing entrance of pollutants or external materials to graphene-Si Schottky junction 333. In an exemplary embodiment, exemplary sample holder 334 may include one or more confining walls, for example, walls 335, 336, 337, and 338 around graphene-Si Schottky junction 333. In an exemplary embodiment, exemplary sample holder 334 may be made of plexiglass including a plastic material made from poly methyl methacrylate.

Referring back to FIG. 1, exemplary system 100 may further include exemplary light source 104 placed above biosensor 102. In an exemplary embodiment, light source 104 may be placed above graphene-semiconductor Schottky junction 128 of biosensor 102. In an exemplary embodiment, light source 104 may include a light emitting device or a light emitting source with a wavelength range of about 300 nm to about 1000 nm. In an exemplary embodiment, light source 104 may include an array of light emitting diodes with a wavelength range of about 300 nm to about 1000 nm. In an exemplary embodiment, light source 104 may include one or more light emitting diodes (LEDs) or one or more light emitting lasers with an emission peak in a range of wavelength of about 350 nm to about 950 nm. In an exemplary embodiment, light source 104 may be configured to irradiate a light beam to an exemplary sample placed and attached on graphene-semiconductor Schottky junction 128. In an exemplary embodiment, an irradiation wavelength of light source 104 may be adjustable using processing unit 108. In an exemplary embodiment, light source 104 may be configured to irradiate the light beam with a wavelength in a range of 500 nm to 900 nm to an exemplary sample placed and attached on graphene-semiconductor Schottky junction 128. In an exemplary embodiment, light source 104 may be configured to irradiate the light beam with a wavelength of 850 nm to an exemplary sample placed and attached on graphene-semiconductor Schottky junction 128. In an exemplary embodiment, light source 104 may include one or more light-emitting diodes (LEDs).

Referring to FIG. 1, exemplary system 100 may include electrical stimulator-analyzer device 106. In an exemplary embodiment, electrical stimulator-analyzer device 106 may be electrically connected to exemplary electrodes 122 and 124 of biosensor 102. In an exemplary embodiment, electrical stimulator-analyzer device 106 may be electrically connected to exemplary electrodes 122 and 124 utilizing three respective electrically conductive lines 114 and 116. In an exemplary embodiment, electrical stimulator-analyzer device 106 may include an electrical voltage generator and an electrical current sensor. In an exemplary embodiment, an exemplary electrical voltage generator of electrical stimulator-analyzer device 106 may be capable of applying an electrical voltage between two exemplary electrodes 122 and 124. In an exemplary embodiment, an exemplary electrical current sensor of electrical stimulator-analyzer device 106 may be capable of measuring an electrical current generated between two exemplary electrodes 122 and 124 responsive to an exemplary applied voltage using an exemplary electrical voltage generator of electrical stimulator-analyzer device 106. In an exemplary embodiment, electrical stimulator-analyzer device 106 may be configured to apply an electrical voltage between two exemplary electrodes 122 and 124 and measure an electrical current generated between two exemplary electrodes 122 and 124 responsive to the applied voltage. In an exemplary embodiment, electrical stimulator-analyzer device 106 may be configured to apply a sweeping set of electrical voltages between two exemplary electrodes 122 and 124 and measure a respective set of electrical currents generated between two exemplary electrodes 122 and 124 responsive to the applied sweeping set of electrical voltages. In an exemplary embodiment, electrical stimulator-analyzer device 106 may be electrically connected to processing unit 108 utilizing at least one of an electrically conductive line 110, a wireless connection, and combinations thereof. In an exemplary embodiment, the wireless connection may include Bluetooth devices or Bluetooth modules, which may be embedded in electrical stimulator-analyzer device 106 and processing unit 108. In an exemplary embodiment, electrical stimulator-analyzer device 106 may be further configured to send the measured electrical current or the measured set of electrical currents to processing unit 108.

In an exemplary embodiment, processing unit 108 may include a memory having processor-readable instructions stored therein and a processor. In an exemplary embodiment, the processor may be configured to access the memory and execute the processor-readable instructions. In an exemplary embodiment, executing the processor-readable instructions by the processor may configures the processor to perform a method. In an exemplary embodiment, the method may include an exemplary method for detecting cancer cells in a sample described herein below.

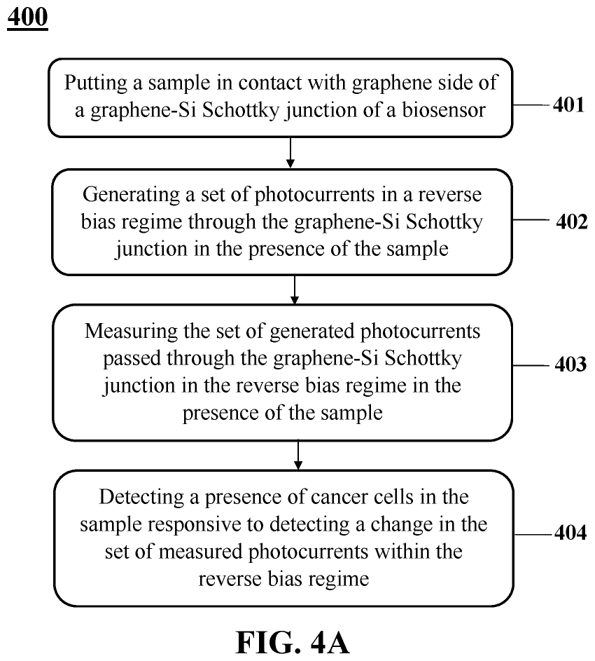

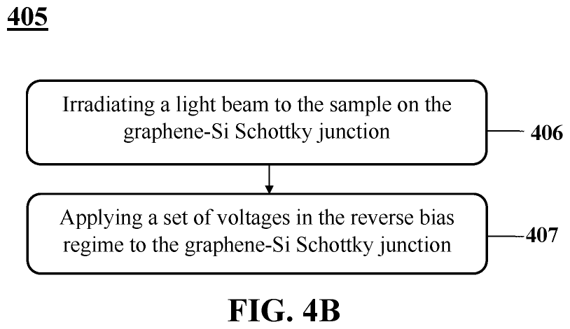

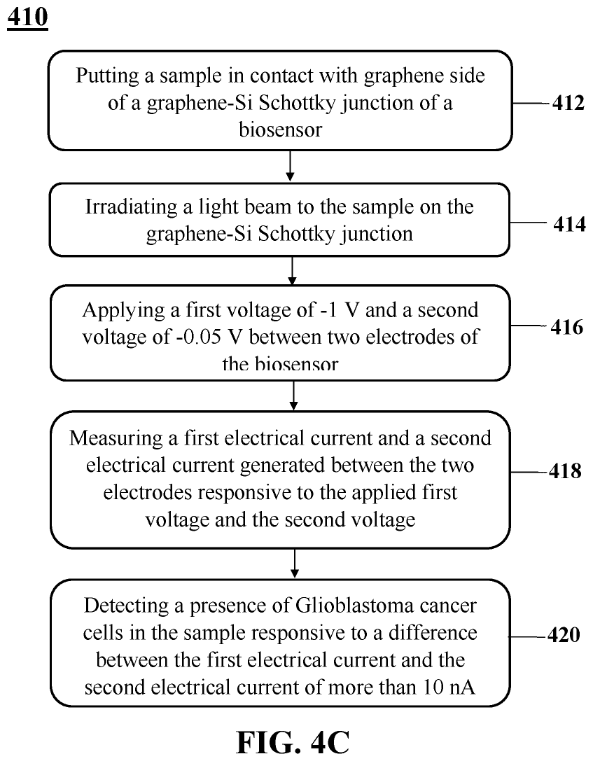

In another general aspect of the present disclosure, an exemplary method for detecting cancer cells in an exemplary sample is described. In an exemplary embodiment, an exemplary method may be carried out utilizing exemplary system 100 described hereinabove. FIG. 4A shows an exemplary flow diagram of an exemplary method 400 for detecting cancer cells in a sample, consistent with one or more exemplary embodiments of the present disclosure. In an exemplary embodiment, one or more steps of exemplary method 400 may be done utilizing an exemplary system similar to exemplary system 100 described hereinabove. Accordingly, exemplary steps of method 400 are described in context of elements of biosensor 102. In an exemplary embodiment, method 400 may include putting an exemplary sample in contact with graphene side 142 of exemplary graphene-semiconductor Schottky junction 128 of biosensor 102 (step 401), generating a set of photocurrents in a reverse bias regime passed through graphene-semiconductor Schottky junction 128 (step 402), measuring an exemplary set of generated photocurrents passed through the graphene-semiconductor Schottky junction 128 in the presence of an exemplary sample (step 403), and detecting a presence of cancer cells in sample 129 if a change is detected in an exemplary set of measured photocurrents within an exemplary reverse bias regime (step 404).

In detail, step 401 may include putting an exemplary sample, for example, sample 129 in contact with graphene side 142 of graphene-semiconductor Schottky junction 128 of biosensor 102. In an exemplary embodiment, exemplary sample 129 may include a sample including biological cells acquired from a person suspected to have cancer. In an exemplary embodiment, exemplary sample 129 may include at least one of a blood sample drawn from a person, a biopsied sample acquired from a person, a sample containing biological cells suspected to be cancerous, and combinations thereof. In an exemplary embodiment, exemplary sample 129 may include a biopsied sample from a mass suspected to be a cancerous tumor in a person's body. In an exemplary embodiment, putting exemplary sample 129 in contact with graphene side 142 of graphene-semiconductor Schottky junction 128 may include dropping or placing exemplary sample 129 inside sample holder 130 on graphene-semiconductor Schottky junction 128. In an exemplary embodiment, putting exemplary sample 129 in contact with graphene side 142 of graphene-semiconductor Schottky junction 128 may lead to attaching/adhering exemplary sample 129 to graphene side 142 of graphene-semiconductor Schottky junction 128 due to dangling bonds of graphene.

Furthermore, step 402 may include generating an exemplary set of photocurrents in a reverse bias regime passed through graphene-semiconductor Schottky junction 128. In an exemplary embodiment, generating an exemplary set of photocurrents through graphene-semiconductor Schottky junction 128 may be carried out utilizing electrical stimulator-analyzer device 106 and light source 104. FIG. 4B shows an exemplary flow diagram of an exemplary method 405 for generating an exemplary set of photocurrents in a reverse bias regime through graphene-semiconductor Schottky junction 128, consistent with one or more exemplary embodiments of the present disclosure. In an exemplary embodiment, method 405 may include irradiating a light beam to exemplary sample 129 on graphene-semiconductor Schottky junction 128 (step 406) and applying a set of voltages in reverse bias regime to graphene-semiconductor Schottky junction 128 (step 407).

In an exemplary embodiment, step 406 of irradiating an exemplary light beam to exemplary sample 129 on graphene-semiconductor Schottky junction 128 may include irradiating an exemplary light beam to biosensor 102. In an exemplary embodiment, irradiating an exemplary light beam to exemplary sample 129 on graphene-semiconductor Schottky junction 128 may include irradiating an exemplary light beam to graphene-semiconductor Schottky junction 128 while exemplary sample 129 being on graphene-semiconductor Schottky junction 128 using exemplary light source 104. In an exemplary embodiment, irradiating an exemplary light beam to graphene-semiconductor Schottky junction 128 with exemplary sample 129 thereon may include irradiating an exemplary light beam with a wavelength in a range of about 500 nm to about 900 nm to graphene-semiconductor Schottky junction 128 with exemplary sample 129 thereon utilizing light source 104.

In an exemplary embodiment, step 407 of applying an exemplary set of voltages in reverse bias regime to graphene-semiconductor Schottky junction 128 may include applying an electrical voltage to graphene-semiconductor Schottky junction 128 in such a way that semiconductor side (Si side) 140 may be held at a higher voltage than graphene side 142. In an exemplary embodiment, step 407 of applying an exemplary set of voltages in reverse bias regime to graphene-semiconductor Schottky junction 128 may include applying a set of voltages in a range of about −1 V to about −0.01 V to graphene-semiconductor Schottky junction 128. In an exemplary embodiment, applying an exemplary set of voltages in reverse bias regime to graphene-semiconductor Schottky junction 128 may include applying an exemplary set of voltages in an exemplary reverse bias regime to graphene-semiconductor Schottky junction 128 in the presence of exemplary sample 129 on graphene-semiconductor Schottky junction 128 while irradiating the light beam to graphene-semiconductor Schottky junction 128. In an exemplary embodiment, applying an exemplary set of voltages in reverse bias regime to graphene-semiconductor Schottky junction 128 may include applying a sweeping range of reverse bias voltages between two exemplary electrodes 122 and 124 utilizing electrical stimulator-analyzer device 106.

In an exemplary embodiment, steps 406 and 407 of irradiating an exemplary light beam to exemplary sample 129 on graphene-semiconductor Schottky junction 128 and applying an exemplary set of voltages in reverse bias regime to graphene-semiconductor Schottky junction 128 may be done concurrently so that an exemplary set of photocurrents passing through graphene-semiconductor Schottky junction 128 may be generated. As used herein, “photocurrent” may refer to a photo-generated electrical current. In an exemplary embodiment, a photo-generated electrical current may be an electrical current with charge carriers generated due to light irradiation by converting photons to electrons. Herein, irradiating a light beam to an exemplary semiconductor material of graphene-semiconductor Schottky junction 128 may lead to generating a photocurrent passed through graphene-semiconductor Schottky junction 128. In detail, when photons hit a depletion region of graphene-semiconductor Schottky junction 128 while conducting step 406 of irradiating an exemplary light beam to exemplary sample 129 on graphene-semiconductor Schottky junction 128, an amount of energy may be absorbed to graphene-semiconductor Schottky junction 128 and may cause excitation of electrons and creation of electron-hole pairs. Exited electrons and created electron-hole pairs are charge carriers which may be separated (electrons go towards Si and holes towards graphene) due to an electric field (applied in step 407) in graphene-semiconductor Schottky junction 128; thereby, leading to generation of photocurrent in graphene-semiconductor Schottky junction 128.

Referring back to FIG. 4A, step 403 may include measuring an exemplary set of generated photocurrents through graphene-semiconductor Schottky junction 128 in an exemplary reverse bias regime in the presence of exemplary sample 129. In an exemplary embodiment, measuring an exemplary set of generated photocurrents through graphene-semiconductor Schottky junction 128 may include measuring a respective set of produced electrical currents between two exemplary electrodes 122 and 124 responsive to an exemplary applied sweeping range of reverse bias voltages utilizing electrical stimulator-analyzer device 106. In an exemplary embodiment, an exemplary respective set of produced electrical currents between two exemplary electrodes 122 and 124 may include an exemplary set of generated photocurrents due to irradiating an exemplary light beam to graphene-semiconductor Schottky junction 128 while applying an exemplary sweeping range of reverse bias voltages to graphene-semiconductor Schottky junction 128. In an exemplary embodiment, applying an exemplary sweeping range of reverse bias voltages between two exemplary electrodes 122 and 124 may include applying a set of voltages in a range of about −1 V to about −0.01 V between two exemplary electrodes 122 and 124.

Additionally, step 404 may include detecting a presence of cancer cells in exemplary sample 129 if a change is detected in an exemplary measured set of generated photocurrents within an exemplary reverse bias regime. In an exemplary embodiment, detecting the presence of cancer cells in exemplary sample 129 may include detecting at least two electrical currents within an exemplary measured set of produced electrical currents being different with each other by a difference magnitude of more than about 10 nA. In an exemplary embodiment, exemplary sample 129 may be detected to be healthy or normal if all electrical current magnitudes of the measured set of produced electrical currents are the same. In an exemplary embodiment, exemplary sample 129 may be detected to be healthy or normal if a difference between magnitudes of each two electrical currents of an exemplary measured set of produced electrical currents is less than about 10 nA.

In an exemplary embodiment, step 407 may include applying two electrical voltages in an exemplary reverse bias regime between two exemplary electrodes 122 and 124; therefore, step 403 may include measuring two respective photocurrents generated responsive to exemplary applied two electrical voltages. Thereafter, detecting a presence of cancer cells in exemplary sample 129 (step 404) may include comparing exemplary two measured photocurrents respective to exemplary two applied electrical voltages with each other and detecting cancer cells in exemplary sample 129 if exemplary two measured photocurrents are different with each other by a value of more than about 10 nA. In an exemplary embodiment, exemplary two applied electrical voltages may include a first voltage of −1 V and a second voltage of −0.05 V.

In an exemplary embodiment, an exemplary method for detecting Glioblastoma cancer cells in an exemplary sample is described. FIG. 4C shows an exemplary flow diagram of exemplary method 410 for detecting Glioblastoma cancer cells in a sample, consistent with one or more exemplary embodiments of the present disclosure. In an exemplary embodiment, one or more steps of exemplary method 410 may be done utilizing exemplary system 100 described hereinabove. Accordingly, exemplary steps of method 410 are described in context of elements of biosensor 102. In an exemplary embodiment, one or more steps of exemplary method 410 may be similar to one or more steps of exemplary method 400 and/or exemplary method 405 described hereinabove. In an exemplary embodiment, method 410 may include putting exemplary sample 129 in contact with graphene side 142 of graphene-semiconductor Schottky junction 128 of biosensor 102 (step 412), irradiating a light beam with a wavelength in a range of about 500 nm to about 900 nm to exemplary sample 129 on graphene-semiconductor Schottky junction 128 (step 414), applying a first voltage of −1 V and a second voltage of −0.05 V between two electrodes 122 and 124 of biosensor 102 (step 416), measuring a first electrical current generated between two electrodes 122 and 124 responsive to the applied first voltage and a second electrical current generated between two electrodes 122 and 124 responsive to the applied second voltage (step 418), and detecting a presence of Glioblastoma cancer cells in exemplary sample 129 if a difference between the first electrical current and the second electrical current is more than about 10 nA (step 420).

In detail, step 412 may include putting exemplary sample 129 in contact with graphene side 142 of graphene-semiconductor Schottky junction 128 of biosensor 102 similar to step 401 of exemplary method 400. In an exemplary embodiment, putting exemplary sample 129 in contact with graphene side 142 of graphene-semiconductor Schottky junction 128 of biosensor 102 may include putting exemplary sample 129 on graphene-semiconductor Schottky junction 128. In an exemplary embodiment, exemplary sample 129 may include a sample drawn from a person suspected to have Glioblastoma cancer.

In an exemplary embodiment, step 414 may include irradiating a light beam with a wavelength in a range of about 500 nm to about 900 nm to exemplary sample 129 put on graphene-semiconductor Schottky junction 128 similar to step 406 of exemplary method 405. In an exemplary embodiment, irradiating an exemplary light beam with a wavelength in a range of about 500 nm to about 900 nm to exemplary sample 129 may include irradiating an exemplary light beam with a wavelength of about 600 nm to exemplary sample 129 put on graphene-semiconductor Schottky junction 128. In an exemplary embodiment, irradiating an exemplary light beam with a wavelength in a range of about 500 nm to about 900 nm to exemplary sample 129 may include irradiating an exemplary light beam with a wavelength of about 850 nm to exemplary sample 129 put on graphene-semiconductor Schottky junction 128.

Furthermore, step 416 may include applying two voltages in a range of bias voltages between two electrodes 122 and 124 of biosensor 102. In an exemplary embodiment, a first voltage of about −1 V and a second voltage of about −0.05 V may be applied between two electrodes 122 and 124 of biosensor 102. In an exemplary embodiment, two electrical currents (photocurrents) may be generated due to the applied first voltage and the applied second voltage while irradiating an exemplary light beam in the presence of exemplary sample 129 on graphene-semiconductor Schottky junction 128. In an exemplary embodiment, step 416 may include an exemplary process similar to step 407 of exemplary method 405.

In an exemplary embodiment, step 418 may include measuring the two generated electrical currents utilizing electrical stimulator-analyzer device 106. In an exemplary embodiment, measuring the two generated electrical currents may include measuring a first electrical current generated between two electrodes 122 and 124 responsive to the applied first voltage of about −1 V and a second electrical current generated between two electrodes 122 and 124 responsive to the applied second voltage of about −0.05 V. In an exemplary embodiment, step 418 may include an exemplary process similar to step 403 of exemplary method 400.

Moreover, step 420 may include detecting a presence of Glioblastoma cancer cells in an exemplary sample if a difference between the measured first electrical current and the measured second electrical current is more than about 10 nA. In an exemplary embodiment, step 420 may include comparing the measured first electrical current with the measured second electrical current by calculating a difference between values of the measured first electrical current and the measured second electrical current and detecting the presence of Glioblastoma cancer cells in an exemplary sample if the measured first electrical current and the measured second electrical current are different with each other by a value of 10 nA or more. Furthermore, step 420 may further include detecting an exemplary sample being normal or healthy if a difference between the measured first electrical current and the measured second electrical current is less than about 10 nA. In an exemplary embodiment, an exemplary sample may be detected to be normal or healthy if the measured first electrical current and the measured second electrical current have the same magnitude.

In an exemplary embodiment, exemplary methods 400, 405, and 410 may be utilized as fast and real-time methods for detecting cancer. In an exemplary embodiment, exemplary methods 400, 405, and 410 may be done in less than about one minute. In an exemplary embodiment, exemplary methods 400, 405, and 410 may be done in less than about 30 seconds.

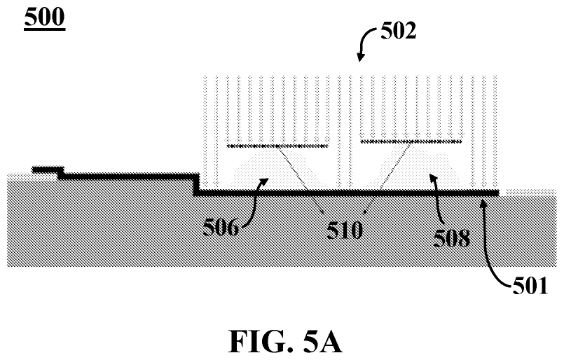

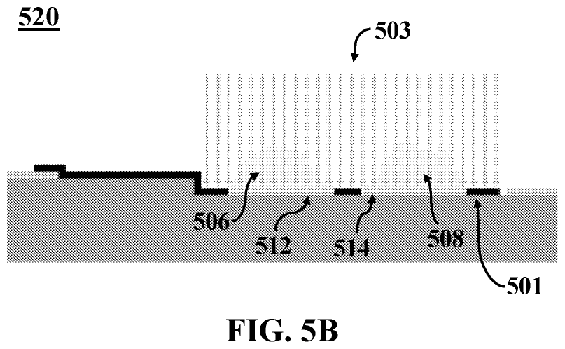

In an exemplary embodiment, exemplary biosensor 102 may be utilized for detecting and differentiating biological cells by recording and monitoring changes in photocurrent generated in graphene-semiconductor Schottky junction 128 of exemplary biosensor 102 in reverse bias. In an exemplary embodiment, changes in photocurrent generated in graphene-semiconductor Schottky junction 128 of exemplary biosensor 102 in reverse bias may be explained and analyzed by an interplay of two different physical mechanisms of operation of exemplary biosensor 102, each more prominent in a particular wavelength range. In an exemplary embodiment, exemplary two different physical mechanisms may include a first mechanism including a shadow effect regime and a second mechanism including a charge transfer effect regime. In an exemplary embodiment, an exemplary first mechanism of shadow effect regime may be activated when a light beam with a wavelength in a first wavelength range is irradiated to biosensor 102, whereas an exemplary second mechanism of charge transfer effect regime may be activated while irradiating a light beam with a wavelength in a second wavelength range to biosensor 102. In an exemplary embodiment, an exemplary second wavelength range may include higher wavelengths than wavelength of an exemplary first wavelength range. In an exemplary embodiment, an exemplary first wavelength range may include wavelength magnitudes of less than a threshold wavelength and an exemplary second wavelength range may include wavelength magnitudes of more than the threshold wavelength. In an exemplary embodiment, an exemplary threshold wavelength may include a wavelength of about 600 nm. In an exemplary embodiment, the threshold wavelength may include a wavelength of 520 nm for Glioblastoma cancer cells. In an exemplary embodiment, an exemplary second wavelength range may include wavelength magnitudes near infrared (IR) window between about 600 nm and 900 nm. FIGS. 5A and 5B show two schematic representations 500 and 520 of exemplary two respective different regimes of operation for exemplary biosensor 102. FIG. 5A shows a schematic representation 500 of an exemplary shadow effect regime of operation of exemplary biosensor 102, consistent with one or more exemplary embodiments of the present disclosure. Moreover, FIG. 5B shows a schematic representation 520 of an exemplary charge transfer effect regime of operation of exemplary biosensor 102, consistent with one or more exemplary embodiments of the present disclosure.

In an exemplary first mechanism of shadow effect regime illustrated in FIG. 5A while irradiating a light beam 502 with a wavelength magnitude in an exemplary first wavelength range below about 600 nm to exemplary graphene/Si Schottky junction 501 (similar to graphene-semiconductor Schottky junction 128 of exemplary biosensor 102), a main mechanism of operation of exemplary biosensor 102 may relate to an exemplary physical obstruction 510 of exemplary graphene/Si Schottky junction 501 made by exemplary adhered cells 506 and 508. In an exemplary embodiment, a “first regime” or “shadow effect regime” refers to a wavelength of irradiation of about 600 nm or less. In detail, light beam 502 with a short wavelength of less than about 600 nm is not capable of passing through cells 506 and 508 adhered to exemplary graphene/Si Schottky junction 501; thereby, exemplary physical obstruction 510 exemplary graphene/Si Schottky junction 501 may be formed. In more details, exemplary physical obstruction 510 may decrease an effective surface area of exemplary graphene/Si Schottky junction 501 for photon absorption; thereby, resulting in decreasing a generated electrical current under illumination in exemplary graphene/Si Schottky junction 501. As used herein, “an effective surface area” of an exemplary graphene/Si Schottky junction (similar to graphene/Si Schottky junction 501) may include a freely exposed surface area of an exemplary graphene/Si Schottky junction to irradiation, for example light irradiation. In detail, in wavelengths of less than 600 nm, exemplary adhered cells 506 and 508 may block an effective surface of an active region in exemplary graphene/Si junction 501, and thus decrease a photocurrent produced in the presence of light irradiation to exemplary graphene/Si junction 501. In details, some percentage of incident photons may be absorbed by exemplary adhered cells 506 and 508 so that absorption percentage in graphene/Si junction 501 may become less than when there is no cell. In more details, photons cannot reach to total surface area of graphene/Si Schottky junction 501 due to screening of graphene/Si junction 501 by exemplary adhered cells 506 and 508, and consequently photon absorption by graphene/Si junction 501 decreases, leading to decreasing photo-generated current through graphene/Si junction 501. In an exemplary embodiment, such screening of graphene/Si junction 501 by exemplary adhered cells 506 and 508 may be caused due to light absorption by exemplary adhered cells 506 and 508. As a result, in an exemplary first wavelength range, a produced photocurrent is lower in the presence of cancer cells 506 and 508 relative to a bare exemplary graphene/Si Schottky junction 501. In an exemplary first regime, physical shape and orientation of cells 506 and 508 on exemplary graphene layer may play the most significant part in generated photocurrent of exemplary biosensor 102. For example, in an exemplary first regime of irradiating a light beam in an exemplary first wavelength range, exemplary biosensor 102 may show a lower photocurrent in the presence of U87 cell lines in comparison with a presence of T98G cell lines adhered to an exemplary graphene/Si Schottky junction 501.

In an exemplary embodiment, an exemplary second mechanism of charge transfer effect regime is illustrated in FIG. 5B while irradiating a light beam 503 with a wavelength magnitude in a second wavelength range more than about 600 nm to exemplary graphene/Si Schottky junction 501 and the start of the near infrared (IR) window in biological tissue. In an exemplary embodiment, “second regime” or “charge transfer effect regime” refers to a wavelength range of irradiation in which exposed cells to irradiation are almost transparent. In an exemplary embodiment, “second regime” or “charge transfer effect regime” refers to a wavelength range of more than about 600 nm. However, an exemplary second mechanism may not be dependent on physical obstruction of an effective surface area of exemplary graphene/Si Schottky junction 501, as an exemplary second wavelength range falls within near infrared window in biological tissues, light may penetrate exemplary adhered cells 506 and 508 and reach exemplary graphene/Si Schottky junction 501. In an exemplary second regime, charge may be transferred between cells 506 and 508 and graphene layer or a field effect of charged outer surface of cells 506 and 508 may locally alter work function of graphene layer by either adding electrons (increasing Fermi energy or lowering the work function) or taking electrons from graphene (lowering the Fermi energy or increasing the work function). Such change in the work function may results in local areas 512 and 514 under exemplary adhered cells 506 and 508 to have lower or higher Schottky barrier heights that either may facilitate or hinder charge transfer between graphene and Si layer. So, exemplary biosensor 102 may also show distinctive electrical behavior in the presence of respective distinct types of cancer cells adhered to exemplary graphene/Si Schottky junction 501 in higher wavelengths. For example, in an exemplary second regime of irradiating a light beam in an exemplary second wavelength range, exemplary biosensor 102 may show a higher photocurrent in the presence of U87 cell lines, while in the presence of T98G cell lines, exemplary photocurrent may remain constant, and lower than a photocurrent of an exemplary bare graphene/Si Schottky junction 501. Accordingly, both exemplary two regimes of shadow effect and charge transfer effect can be used to differentiate between various cell lines using exemplary biosensor 102. While an exemplary shadow effect in shorter wavelengths may give a measure of geometrical shape of adhered cells to graphene monolayer, as a produce photocurrent depends on physical obstruction of active area of an exemplary Schottky junction, an exemplary charge transfer effect can yield a measure of electrical interaction of cells with graphene monolayer and local changes in Fermi level of graphene. Therefore, both shadow effect and charge transfer effect may be utilized in exemplary biosensor 102; allowing for exemplary biosensor 102 being sensitive to presence of cells and at the same time, being selective (based on cell sizes, and physical properties, and electrical interactions of cells with graphene).

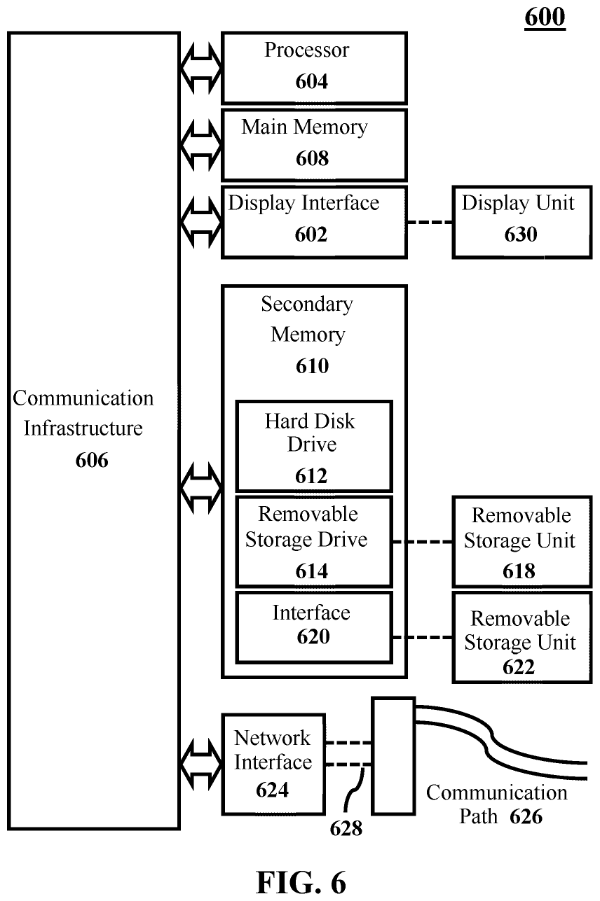

FIG. 6 shows an example computer system 500 in which an embodiment of the present disclosure, or portions thereof, may be implemented as computer-readable code, consistent with one or more exemplary embodiments of the present disclosure. For example, computer system 600 may include an example of processing unit 108 illustrated in FIG. 1, steps 402-404 of exemplary method 400 presented in FIG. 4A, and steps 414-420 of exemplary method 410 presented in FIG. 4B, may be implemented in computer system 600 using hardware, software, firmware, tangible computer readable media having instructions stored thereon, or a combination thereof and may be implemented in one or more computer systems or other processing systems. Hardware, software, or any combination of such may embody any of the modules and components in FIGS. 1, 4A and 4B.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the present disclosure is described in terms of this example computer system 600. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 604 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 604 may be connected to a communication infrastructure 606, for example, a bus, message queue, network, or multi-core message-passing scheme.

In an exemplary embodiment, computer system 600 may include a display interface 602, for example a video connector, to transfer data to a display unit 630, for example, a monitor. Computer system 600 may also include a main memory 608, for example, random access memory (RAM), and may also include a secondary memory 610. Secondary memory 610 may include, for example, a hard disk drive 612, and a removable storage drive 614. Removable storage drive 614 may include a floppy disk drive, a magnetic tape drive, an optical disk drive, a flash memory, or the like. Removable storage drive 614 may read from and/or write to a removable storage unit 618 in a well-known manner. Removable storage unit 618 may include a floppy disk, a magnetic tape, an optical disk, etc., which may be read by and written to by removable storage drive 614. As will be appreciated by persons skilled in the relevant art, removable storage unit 618 may include a computer usable storage medium having stored therein computer software and/or data.

In alternative embodiments, secondary memory 610 may include other similar means for allowing computer programs or other instructions to be loaded into computer system 600. Such means may include, for example, a removable storage unit 622 and an interface 620. Examples of such means may include a program cartridge and cartridge interface (such as that found in video game devices), a removable memory chip (such as an EPROM, or PROM) and associated socket, and other removable storage units 622 and interfaces 620 which allow software and data to be transferred from removable storage unit 622 to computer system 600.

Computer system 600 may also include a communications interface 624. Communications interface 624 allows software and data to be transferred between computer system 600 and external devices. Communications interface 624 may include a modem, a network interface (such as an Ethernet card), a communications port, a PCMCIA slot and card, or the like. Software and data transferred via communications interface 624 may be in the form of signals, which may be electronic, electromagnetic, optical, or other signals capable of being received by communications interface 624. These signals may be provided to communications interface 624 via a communications path 626. Communications path 626 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 618, removable storage unit 622, and a hard disk installed in hard disk drive 612. Computer program medium and computer usable medium may also refer to memories, such as main memory 608 and secondary memory 610, which may be memory semiconductors (e.g. DRAMs, etc.).

Computer programs (also called computer control logic) are stored in main memory 608 and/or secondary memory 610. Computer programs may also be received via communications interface 624. Such computer programs, when executed, enable computer system 600 to implement different embodiments of the present disclosure as discussed herein. In particular, the computer programs, when executed, enable processor device 604 to implement the processes of the present disclosure, such as the operations in methods 400 and 410 illustrated by FIGS. 4A and 4B, discussed above. Accordingly, such computer programs represent controllers of computer system 600. Where an exemplary embodiment of method 200 is implemented using software, the software may be stored in a computer program product and loaded into computer system 600 using removable storage drive 614, interface 620, and hard disk drive 612, or communications interface 624.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Biosensor Fabrication

In this example, an exemplary biosensor similar to biosensor 102 was fabricated via a process similar to a process according to exemplary method 200 described hereinabove and schematically illustrated in FIGS. 3A-3I. An n-type Si wafer with approximate dimensions of 1 cm in 2 cm was used as an exemplary substrate. In a first step, the substrate was thoroughly cleaned using RCA#1 method (with NH₄OH: H₂O₂: H₂O solution and a volume ratio of 1:1:5), rinsed in deionized (DI) water, and dried. An insulating oxide layer was then formed on the substrate using an open chemical vapor deposition (CVD) quartz furnace similar to step 202 of exemplary method 200. The substrate was placed in the furnace, and temperature was slowly ramped up to 1100° C. and held there for oxidation under a constant flow of O₂ gas. After approximately 40 minutes, a 500 nm thick silicon oxide (SiO₂) layer was formed on the Si substrate. In the next step similar to step 204 of exemplary method 200, half of this oxide layer was removed using 10% HF acid solution, while the other half was covered with a cured negative photoresist. This photoresist layer was then etched away with acetone. To form metallic contacts similar to step 206 of exemplary method 200, two square windows with approximate dimensions of 3 mm in 3 mm were formed using standard photolithography, where one window was placed on the oxide layer and the other on the bare Si substrate. Next, the substrate was placed in a physical vapor deposition (PVD) chamber and a 100 nm gold (Au) thin film was deposited on the substrate. The substrate was then rinsed in acetone to clear away the remaining photoresist layer, leaving only two metallic Au contacts. Similar to step 208 of exemplary method 200, graphene mono-layers were deposited on 18 μm thick Cu foils, and then coated with a thin layer of polymethylmethacrylate (PMMA). Graphene layer of the appropriate proportions was cut from Cu/graphene/PMMA foil using standard scissors. This layer was then transferred into a beaker containing FeC13 solution, where it floats on top of the solution as the Cu is dissolved away. The layer was then fished out using an RCA#1 cleaned glass slide into a beaker containing distilled water. The last step was repeated several times to ensure that there was no residue left from the FeCl₃ solution. In the final step, the graphene/PMMA layer was dip coated on the Si/SiO₂/Au layer in a way that the graphene layer came into contact with the Au contact on the SiO₂ layer on one side and the Au contact on n-type Si on the other side. After this step, the substrate was left to dry out to improve the adhesion of the graphene to the substrate. Finally, the PMMA layer was dissolved using acetone, and the sensor was annealed in an oven at 150° C. for two hours.

### Example 2: Cancer Cells Detection via Monitoring Photocurrent of Graphene/Silicon Schottky Junction of an Exemplary Biosensor

In this example, a method similar to exemplary methods 400 and 410 utilizing a fabricated biosensor (similar to biosensor 102) of Example 1 hereinabove was used to detect and differentiate Glioblastoma cancer cells from each other and from normal (healthy) cells. Glioma cell line U87MG (IBRC C10982), Glioma cell line T98G, and healthy human fibroblast cells were provided and STR DNA Profiling Analysis was performed to authenticate of Glioma cell lines. Cells were cultured in Dulbecco's modified Eagle's medium (DMEM) supplemented with 100 U/ml penicillin and 100 g/ml streptomycin plus 10% fetal bovine serum (FBS). Cells were incubated at 37° C. in a humidified atmosphere containing 5% CO₂ until 90% confluency was reached. In order to coat exemplary fabricated biosensors similar to sensor 102 by cells similar to each of steps 401 and 412, approximately 1.2×10⁶ of each cell lines and human fibroblast were suspended in 1 ml phosphate buffer saline and poured gently on the biosensors which were placed on the bottom of 6 respective well plates in duplicate. The plates were incubated at 37° C. in a humidified atmosphere containing 5% CO₂. Controls of cell growth and confluency were wells which were coated by each of the cell lines and human fibroblast. By the time the control wells have reached 90% confluency, the cell coated biosensors were removed from each well and were used in photocurrent tests. Silicon epoxy containers were used to isolate exemplary graphene layers of the biosensors and to act as a barrier to hold an exemplary solution containing the cells. All biosensors were characterized in dark and also under irradiation by different wavelengths of light before the addition of cells. The cells were added to the epoxy containers that were created to hold the cell solution, and during tests, these wells were filled with PBS solutions. These tests were carried out similar to steps 404-408 and/or 414-420 and repeated in the presence of different cell lines, and changes in photocurrent were recorded for comparison. High quality commercial 3W LEDs were used as light sources with peak intensities in 380 nm, 425 nm, 520 nm, 620 nm, 740 nm and 850 nm for irradiating a light beam to cells adhered to an exemplary graphene/Si Schottky junction of an exemplary fabricated biosensor similar to each of steps 401 and 412.

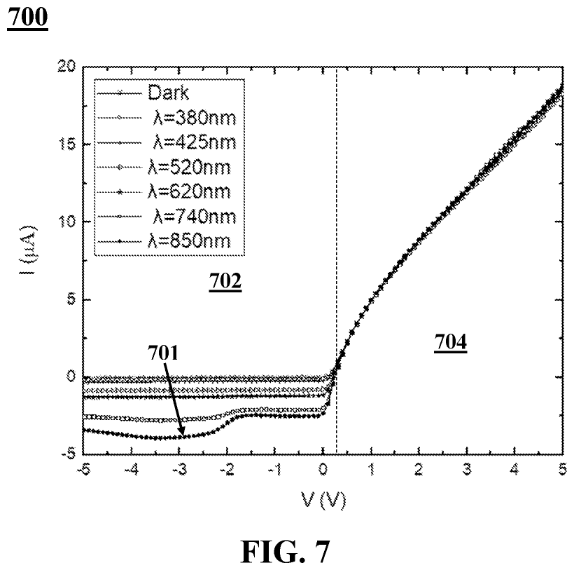

FIG. 7 shows graph 700 that illustrates current-voltage characteristics of exemplary fabricated graphene/Si Schottky junction in dark and under illumination by different wavelengths of light, including 380 nm, 425 nm, 520 nm, 620 nm, 740 nm, and 850 nm with the same intensity of about 200 μW/cm², consistent with one or more exemplary embodiments of the present disclosure. It may be seen that under illumination in reverse bias (part 702 of diagram), exemplary biosensor shows a significant increase in current by increasing irradiated light wavelength in a direction illustrated by arrow 701 from 380 nm to 850 nm, while forward bias (part 704 of diagram) remains largely unchanged. A change in the reverse bias current increases as wavelength increases towards red and IR region (for λ more than 650 nm). Accordingly, cancer diagnostic tests were confined to a reverse bias regime of an exemplary graphene/Si Schottky contact, where it acts as a broadband photodetector, and changes in photocurrent were recorded.

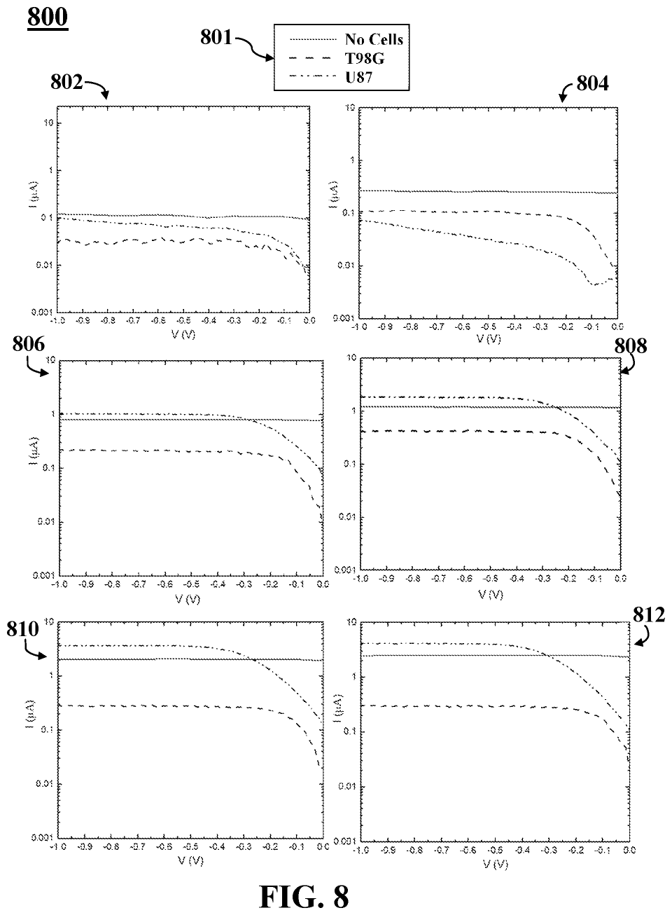

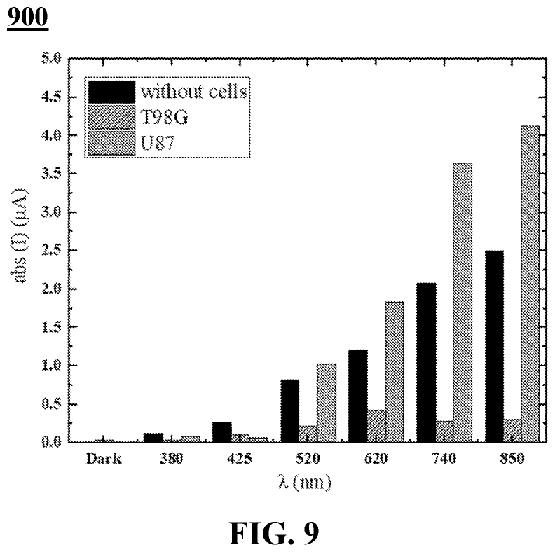

FIG. 8 shows a set of diagrams 800 illustrating current-voltage characteristics of an exemplary biosensor with and without T98G and U87 cell lines as specified in top guide 801, in dark and under light illumination with different wavelengths of light of 380 nm (diagram 802), 425 nm (diagram 804), 520 nm (diagram 806), 620 nm (diagram 808), 740 nm (diagram 810), and 850 nm (diagram 812), consistent with one or more exemplary embodiments of the present disclosure. As may be seen, in wavelengths longer than 520 nm, a better distinction between cells may be obtained. For easier comparison of data represented in FIG. 8, FIG. 9 shows a graph 900 illustrating reverse photocurrent of exemplary biosensor in the absence and presence of different cancer cell lines for different wavelengths at diode voltage (V_{D}) of −1 V, consistent with one or more exemplary embodiments of the present disclosure. It may be seen that below 520 nm for wavelengths (λ) of 425 nm or less, photocurrent is lower in the presence of both cell lines relative to bare Schottky junction without any cells. Whereas, above 520 nm and the start of the near IR window in biological tissue, exemplary biosensor shows a higher photocurrent in the presence of U87 cell lines, while in the presence of T98G cell lines, the photocurrent seems to remain more or less constant, and lower than the photocurrent of the bare Schottky junction. Such changes and differences in reverse regime behavior may be used to differentiate between the two cell lines U87 and T98G. As a result, for U87 cells and T98G cells, in a shadow effect regime, there is a greater current decrease for U87 cells in comparison with T98G cells. As wavelength increases and charge transfer effect becomes more dominant in IR window, these two cells switch places and lower reverse current belongs to T98G cells. This means that T98G cells have induced depletion regions on graphene monolayer and therefore, created higher Schottky barriers in graphene monolayer. Specifically regarding FIGS. 5A and 5B described hereinabove, in an exemplary second regime of irradiating a light beam in an exemplary second wavelength range, exemplary biosensor 102 may show a higher photocurrent in the presence of U87 cell lines, while in the presence of T98G cell lines, exemplary photocurrent may remain constant, and lower than a photocurrent of an exemplary bare graphene/Si Schottky junction.

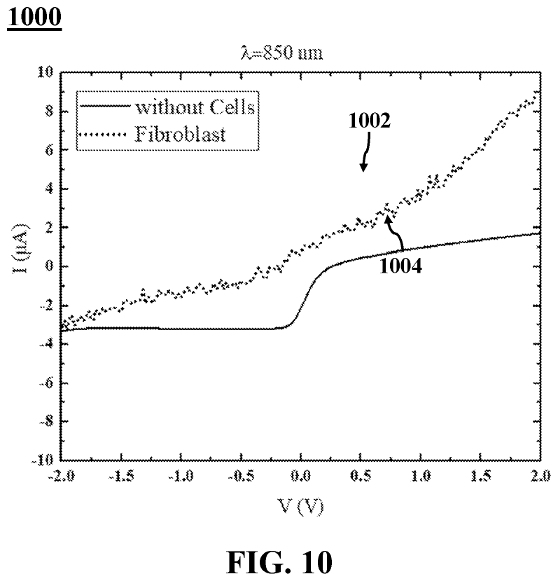

Healthy human fibroblast cells were also tested to determine behavior of an exemplary biosensor in the presence of healthy cells. FIG. 10 shows a graph 1000 illustrating I-V characteristics of an exemplary graphene/Si Schottky junction (diode) under illumination with wavelength of 850 nm in the presence of human fibroblast cells (curve 1002) and in a bare mode without any cells thereon (curve 1004), consistent with one or more exemplary embodiments of the present disclosure. As seen in FIG. 10, in the presence of human fibroblast cells, there is a drastic change in current-voltage characteristic curve of an exemplary biosensor and a rectifying Schottky junction behavior is almost completely diminished (curve 1002) in comparison with a rectifying behavior of exemplary biosensor without cells (curve 1004). This is further confirmed in relative change of photocurrent, where an increasing behavior may be seen for electrical currents generated within reverse bias regime in the presence of healthy human fibroblast cells (curve 1002) in comparison with an approximately constant electrical current within reverse bias regime in the presence of cancer cells (FIGS. 7 and 8) or in the absence of any cells (curve 1004).

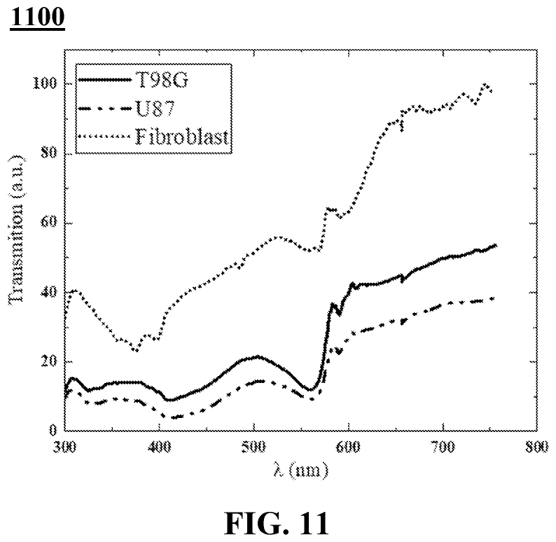

As described hereinabove referring to FIG. 4, a diagnostic behavior of photoelectrical properties of an exemplary biosensor in the presence of cancer cells may be monitored in an exemplary second range of wavelength magnitudes more than about 600 nm. FIG. 11 shows a graph 1100 illustrating UV-Visible transmission spectra of PBS solutions containing T98G, U87, and human fibroblast cell lines, consistent with one or more exemplary embodiments of the present disclosure. As may be seen from FIG. 11, there is a sudden increase in light transmission for both T98G and U87 cells for wavelengths larger than 600 nm in an exemplary second regime or charge transfer effect regime (the start of the near IR window). There appears to be no prominent qualitative difference between the transmission spectra of the two T98G and U87 cancer cell lines. Therefore, the main mechanism behind different photoresponses of an exemplary biosensor in the presence of these two Glioblastoma cell lines is due to an electrical interaction between the cells and the graphene monolayer.

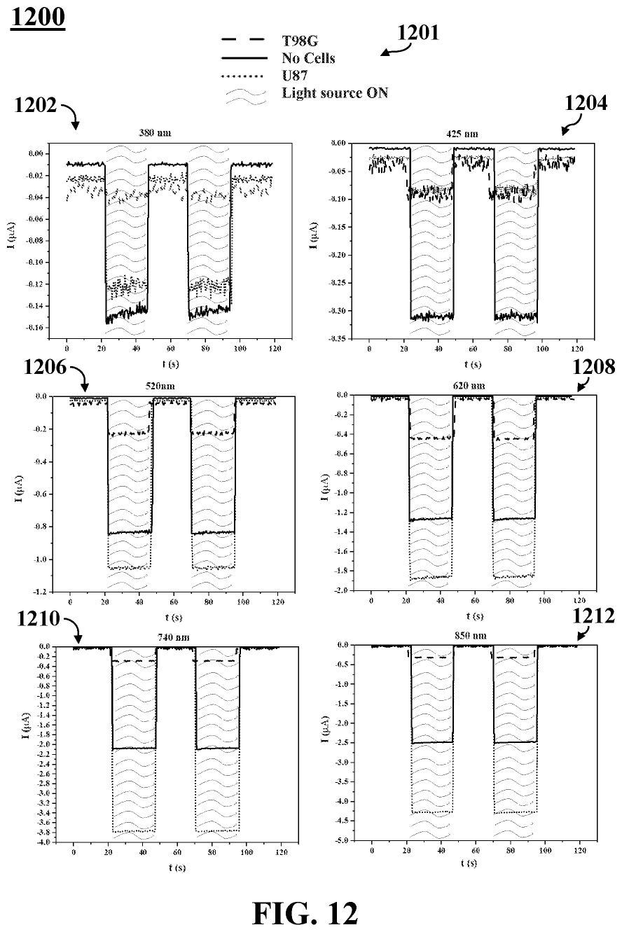

To better demonstrate sensitivity and selectivity of an exemplary biosensor, amperometric tests were performed under a reverse bias voltage of −1 V. FIG. 12 shows a set of diagrams 1200 illustrating current versus time (I-t) characteristics with and without T98G, U87, fibroblast cells in dark and under illumination with lights of different wavelengths of 380 nm (diagram 1202), 425 nm (diagram 1204), 520 nm (diagram 1206), 620 nm (diagram 1208), 740 nm (diagram 1210), and 850 nm (diagram 1212) by applying V_{D} of −1 V as specified in top guide 1201, consistent with one or more exemplary embodiments of the present disclosure. An exemplary biosensor with no cells, and exemplary biosensors with U87 and T98G cell lines behave as a standard Schottky junction with small dark currents and a high photocurrent.

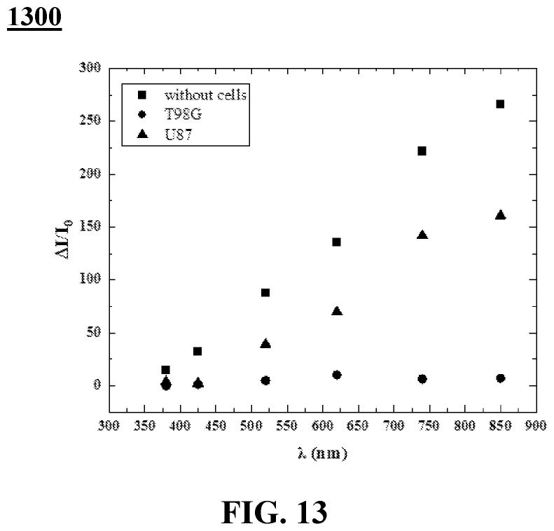

To better quantify the results, relative photocurrent change (RPC) in the reverse bias is plotted in FIG. 13. FIG. 13 shows a chart 1300 illustrating relative change of photocurrent versus wavelength with and without different cell lines at V_{D} of −1 V, consistent with one or more exemplary embodiments of the present disclosure. It may be seen that in the absence of cells and in the presence of U87 cell line, there is an increase in the RPC with increasing wavelength. However, the T98G cell line shows an increase in the RPC from shorter wavelengths to 520 nm, but starts to show a decrease in the RPC from 520 nm to 850 nm. In a context of a hypothesized shadow effect and charge transfer effect, the initial increase of the RPC from 380 nm to 520 nm is expected due to the shadow effect of both cell lines. This increase in RPC is a part of the signature behavior of the broadband Si/graphene Schottky junction. However, the subsequent decline of the RPC in the case of T98G cell line, as the wavelength enters into near IR biological window, suggests that these cells create depletion regions with higher Schottky barrier heights. Therefore, although the light in the IR window reaches the active area below the adhered cells, the high Schottky barriers there effectively prohibit any electron emission from graphene to Si.

On the other hand, the U87 cell line in the IR window shows a marked increase in RPC relative to the device with T98G cells. This suggests the existence of local regions with lower Schottky barriers where the cells have adhered to the graphene. This increases the electron emission from graphene to Si in these regions. The different behavior of the two Glioblastoma cancer cell lines can be used to identify and differentiate between U87 and T98G cell lines.

It should also be noted that the T98 cell line comes from a 61 years old male patient, which has no tumorigenic ability. Tumorigenicity is the tendency of cultured cells to develop benign or malignant growing tumors when injected to an immunologically unresponsive animal. U87 cell line is reported to come from a male patient of unknown age, which has tumorigenic ability. U87 cells were significantly more invasive, compared to the T98G cell line.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments. This is for purposes of streamlining the disclosure, and is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

While various embodiments have been described, the description is intended to be exemplary, rather than limiting and it will be apparent to those of ordinary skill in the art that many more embodiments and embodiments are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

What is claimed is:

1. A system for detecting Glioblastoma cancer cells, comprising:
a biosensor, comprising:
a semiconductor layer comprising a silicon (Si) wafer;
an electrically passivating layer, the electrically passivating layer comprising a silicon dioxide (SiO₂) layer coated on a first portion of the Si wafer;
two electrodes, comprising:
a first electrode deposited on a second portion of the Si wafer; and
a second electrode deposited on the SiO₂ layer;
a graphene layer coated on parts of the Si wafer, the SiO₂ layer, and the second electrode forming a graphene-Si Schottky junction between the Si wafer and the graphene layer, a first side of the graphene layer being in contact with the Si wafer and a second side of the graphene layer being in contact with the second electrode, the graphene-Si Schottky junction configured to receive a sample thereon;
a light source placed above the biosensor, the light source comprising a light emitting device with a wavelength range of 300 nm to 1000 nm, the light source configured to irradiate a light beam to the graphene-Si Schottky junction with the sample thereon;
an electrical stimulator-analyzer device electrically connected to the two electrodes of the biosensor, the stimulator-analyzer device comprising:
an electrical voltage generator configured to apply a voltage between the two electrodes; and
an electrical current sensor configured to measure a produced electrical current between the two electrodes responsive to the applied voltage; and
a processing unit electrically connected to the electrical stimulator-analyzer device and the light source, the processing unit comprising:
a memory having processor-readable instructions stored therein; and
a processor configured to access the memory and execute the processor-readable instructions, which, when executed by the processor configures the processor to perform a method, the method comprising:
irradiating, utilizing the light source, a light beam with a wavelength in a range of 500 nm to 900 nm to the graphene-Si Schottky junction with the sample placed thereon;
applying, utilizing the electrical stimulator-analyzer device, a first voltage of −1 V and a second voltage of −0.05 V between the two electrodes while irradiating the light beam to the graphene-Si Schottky junction with the sample placed thereon;
measuring, utilizing the electrical stimulator-analyzer device, a first electrical current generated between the two electrodes responsive to the applied first voltage and a second electrical current generated between the two electrodes responsive to the applied second voltage; and
detecting a presence of Glioblastoma cancer cells in the sample responsive to detecting a difference between the first electrical current and the second electrical current being more than 10 nA.

2. The system of claim 1, wherein irradiating the light beam to the graphene-Si Schottky junction with the sample placed thereon comprises irradiating the light beam with a wavelength of 850 nm to the graphene-Si Schottky junction with the sample placed thereon.

3. The system of claim 1, wherein detecting the presence of Glioblastoma cancer cells in the sample further comprises differentiating between T98G Glioblastoma cells and U87 Glioblastoma cells in the sample, comprising:
detecting a presence of T98G Glioblastoma cells in the sample responsive to detecting the first electrical current being less than 1 μA; or
detecting a presence of T98G Glioblastoma cells in the sample responsive to detecting the first electrical current being more than 1 μA.

4. The system of claim 1, wherein the sample comprises a biological sample containing biological cells acquired from a person suspected to have Glioblastoma cancer.

5. The system of claim 4, wherein the sample comprises a biopsied sample from a cancer-suspicious mass in body of the person.

6. The system of claim 1, wherein each of the two electrodes comprises a gold (Au) film with a thickness in a range of 50 nm to 200 nm.

7. The system of claim 1, wherein the graphene layer comprises a monolayer graphene film.

8. The system of claim 1, wherein the system further comprises a sample holder placed around the graphene-Si Schottky junction, comprising one or more sidewalls enclosing an area of the graphene-Si Schottky junction with the sample placed thereon, the sample holder being configured to:
keep the sample on surface of the graphene-Si Schottky junction;
prevent the sample from flowing out of the graphene-Si Schottky junction; and
prevent entrance of pollutants or external materials to the graphene-Si Schottky junction.

9. A system for detecting cancer cells, comprising:
a biosensor, comprising:
a semiconductor layer;
an electrically passivating layer coated on a first portion of the semiconductor layer;
two electrodes, comprising:
a first electrode deposited on a second portion of the semiconductor layer; and
a second electrode deposited on the electrically passivating layer;
a graphene layer coated on parts of the semiconductor layer, the electrically passivating layer, and the second electrode forming a graphene-semiconductor Schottky junction between the semiconductor layer and the graphene layer, a first side of the graphene layer being in contact with the semiconductor layer and a second side of the graphene layer being in contact with the second electrode, the graphene-semiconductor Schottky junction being configured to receive a sample thereon;
a light source placed above the biosensor, the light source comprising a light emitting device with a wavelength range of 300 nm to 1000 nm, the light source configured to irradiate a light beam to the graphene-semiconductor Schottky junction with the sample thereon;
an electrical stimulator-analyzer device electrically connected to the two electrodes of the biosensor, the stimulator-analyzer device comprising:
an electrical voltage generator configured to apply a sweeping range of reverse bias voltages between the two electrodes; and
an electrical current sensor configured to measure a set of produced electrical currents between the two electrodes responsive to the applied sweeping range of reverse bias voltages; and
a processing unit electrically connected to the electrical stimulator-analyzer device and the light source, the processing unit comprising:
a memory having processor-readable instructions stored therein; and
a processor configured to access the memory and execute the processor-readable instructions, which, when executed by the processor configures the processor to perform a method, the method comprising:
generating a set of photocurrents in a reverse bias regime through the graphene-semiconductor Schottky junction with the sample placed thereon, comprising:
irradiating, utilizing the light source, a light beam to the graphene-semiconductor Schottky junction with the sample placed thereon; and
applying, utilizing the electrical stimulator-analyzer device, a sweeping range of reverse bias voltages between the two electrodes while irradiating the light beam;
measuring, utilizing the electrical stimulator-analyzer device, the set of generated photocurrents through the graphene-semiconductor Schottky junction in the reverse bias regime in the presence of the sample; and
detecting a presence of cancer cells in the sample responsive to detecting a change in the measured set of the generated photocurrents within the reverse bias regime.

10. The system of claim 9, wherein irradiating the light beam to the graphene-semiconductor Schottky junction with the sample placed thereon comprises irradiating the light beam with a wavelength in a range of 500 nm to 900 nm to the graphene-semiconductor Schottky junction with the sample placed thereon.

11. The system of claim 9, wherein detecting the presence of cancer cells in the sample comprises detecting at least two photocurrent values of the measured set of the generated photocurrents being different with each other by a difference magnitude of more than 10 nA.

12. The system of claim 9, wherein applying the sweeping range of reverse bias voltages between the two electrodes comprises applying a set of voltages in a range of −1 V to −0.01 V between the two electrodes.

13. The system of claim 9, wherein the sample comprises a biological sample containing biological cells acquired from a person suspected to have cancer.

14. The system of claim 14, wherein the sample comprises a biopsied sample from a cancer-suspicious mass in body of the person.

15. The system of claim 9, wherein:
the semiconductor layer comprises a n-type silicon (Si) wafer with a thickness of 500 μm; and
the electrically passivating layer comprises a silicon dioxide (SiO₂) layer with a thickness in a range of 200 nm to 1 μm.

16. The system of claim 9, wherein each of the two electrodes comprises a gold (Au) film with a thickness in a range of 50 nm to 200 nm.

17. The system of claim 9, wherein the graphene layer comprises a monolayer graphene film.

18. The system of claim 9, wherein the method is conducted in less than 30 seconds.

19. The system of claim 9, wherein the system further comprises a sample holder placed around the graphene-semiconductor Schottky junction, the sample holder comprising one or more sidewalls enclosing an area of the graphene-semiconductor Schottky junction with the sample placed thereon, the sample holder being configured to:
keep the sample on surface of the graphene-semiconductor Schottky junction;
prevent the sample from flowing out of the graphene-semiconductor Schottky junction; and
prevent entrance of pollutants or external materials to the graphene-Si Schottky junction.
